(19) United States
(12) Patent Application Publication
(10) Pub. No.: US 2026/0253740 A1
(43) Pub. Date: Aug. 27, 2026

(54) SYSTEM

(71) Applicant: SoftBank Group Corp., Tokyo (JP)

(72) Inventor: Yuya OKAWA, Tokyo (JP)

(73) Assignee: SoftBank Group Corp., Tokyo (JP)

(21) Appl. No.: 19/534,809

(22) Filed: Feb. 10, 2026

(30) Foreign Application Priority Data
Feb. 21, 2025 (JP) 2025-027031

(51) Int. Cl. G16H 50/30; G16H 20/70; G16H 50/20

(52) U.S. Cl. CPC G16H 50/30; G16H 20/70; G16H 50/20

(57) ABSTRACT

The system according to the embodiment comprises an analysis unit, a detection unit, and a diagnosis unit. The analysis unit reads internal communications and analyzes words or surrounding context. The detection unit detects statements with potential risk based on the content analyzed by the analysis unit and accumulates them in a database. The diagnosis unit performs intellectual personality diagnosis and categorizes employees based on the data accumulated by the detection unit.

### CROSS-REFERENCE TO RELATED APPLICATION

The present application claims priority to and incorporates by reference the entire contents of Japanese Patent Application No. 2025-027031 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, statements with potential risk in internal communications have not been sufficiently detected, nor has personality diagnosis of employees been adequately performed, leaving room for improvement.

### SUMMARY OF THE INVENTION

The system according to the embodiment comprises an analysis unit, a detection unit, and a diagnosis unit. The analysis unit reads internal communications and analyzes words or surrounding context. The detection unit detects statements with potential risk based on the content analyzed by the analysis unit and accumulates them in a database. The diagnosis unit performs intellectual personality diagnosis and categorizes employees based on the data accumulated by the detection unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

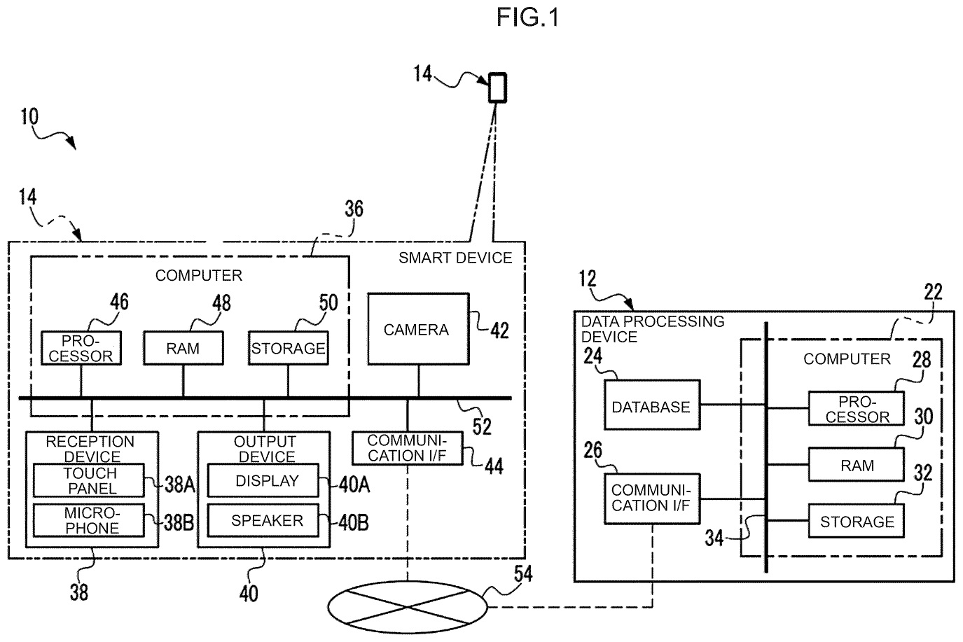

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

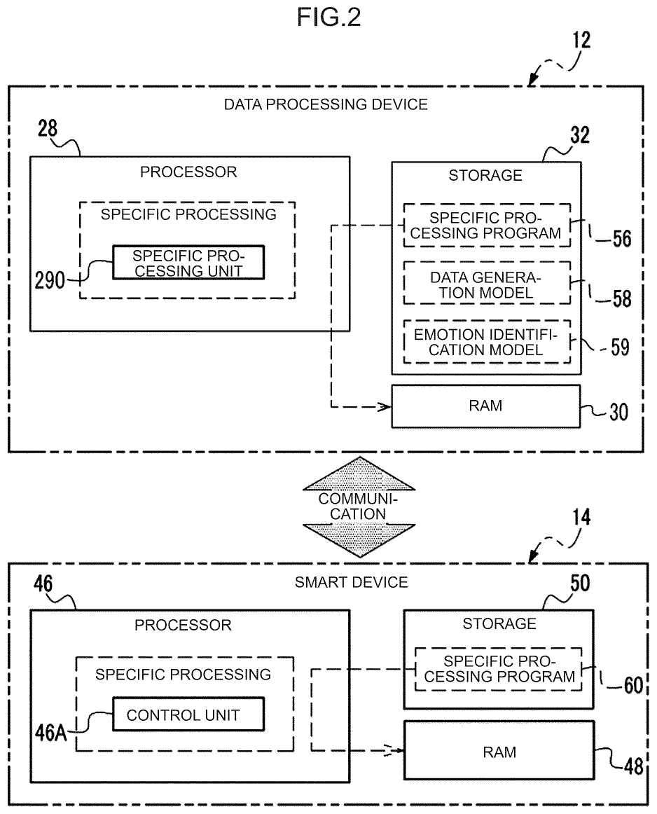

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The system according to the embodiment of the present invention is a system in which AI reads and analyzes internal communications of employees on the system. This system automatically reads internal communications of employees by AI and analyzes words and surrounding context. Next, the AI detects statements with potential risk (power harassment) and accumulates them in a database. Furthermore, the AI performs intellectual personality diagnosis based on daily statements and categorizes employees. With this system, it is possible to prevent harassment in advance and automate the aggregation of human resource information, thereby improving the working environment of the company. For example, an analysis unit is provided that reads internal communications and analyzes words and surrounding context. The analysis unit analyzes the content of employee statements and checks whether specific words or phrases are included. Next, a detection unit is provided that detects statements with potential risk based on the content analyzed by the analysis unit and accumulates them in a database. The detection unit detects aggressive statements and accumulates them in the database. Furthermore, a diagnosis unit is provided that performs intellectual personality diagnosis based on daily statements and categorizes employees. The diagnosis unit analyzes the content and tone of employee statements and diagnoses the personality and intellectual characteristics of the employee. By integrating these functions, a system that automates the prevention of harassment and the aggregation of human resource information is completed. As a result, the system can automate the prevention of harassment and the aggregation of human resource information. Specifically, the system is equipped with a data acquisition module that collects various internal communication data such as internal chat, email, and voice conversation logs. The data acquisition module accepts input such as text data (e.g., UTF-8 encoded string arrays, up to 4096 tokens per sample), audio data (e.g., WAV format sampled at 16 kHz, up to 60 seconds per utterance), and image data (e.g., PNG format screenshots, 1280×720 pixels per image). These data are preprocessed by a preprocessing unit for noise removal, tokenization, speech-to-text conversion (ASR), removal of unnecessary words, and normalization. The analysis unit uses natural language processing models such as Transformer-based large language models, bidirectional LSTM, and convolutional neural networks to extract word embedding vectors (e.g., 768-dimensional vectors of BERT) and contextual features. Examples of input to the analysis unit include text data in which a supervisor says to a subordinate, “You are useless,” and utterance records extracted from audio data during meetings. The analysis unit outputs an aggressive word list (e.g., [‘useless’, ‘incompetent’, ‘fool’]) and context-dependent risk scores (e.g., 0.85/1.0) from these inputs. The detection unit receives the output of the analysis unit and performs threshold judgment (e.g., detection when the risk score is 0.7 or higher) and rule-based filtering (e.g., only statements between specific positions), and records the detection results (e.g., risk statement label, speaker ID, timestamp, risk score) as structured data in the database. Examples of output from the detection unit include “Statement ID: 12345, Risk Statement: True, Score: 0.92, Speaker: Employee A, Date: 2024-06-01 10:15.” The diagnosis unit inputs the statement history and tone information accumulated by the detection unit as time-series vectors (e.g., emotion score arrays for each statement, length 30), and uses a personality diagnosis model (e.g., multilayer perceptron for Big Five personality trait estimation, output: [Extraversion: 0.7, Agreeableness: 0.4, Conscientiousness: 0.8, Neuroticism: 0.2, Openness: 0.6]) to classify employees into multiple intellectual personality categories. Examples of output from the diagnosis unit include “Employee A: High Extraversion, Low Agreeableness, Intellectual Trait: Logical Type.” These outputs are automatically linked to human resource management systems and harassment prevention dashboards and are utilized for early intervention by administrators and planning of organizational improvement measures. Unlike conventional human visual audits and questionnaire tabulation, this system uses computer-specific unconventional methods such as automatic analysis in high-dimensional feature space, hybrid judgment of rule-based and machine learning, and automatic detection of time-series changes, thereby achieving significant improvements in processing speed, reduction of false detection rate, and efficiency in data management. Specific application fields include corporate harassment prevention, appropriate allocation of human resources, healthy internal communication, risk management, and employee mental health support.

The system according to the embodiment comprises an analysis unit, a detection unit, and a diagnosis unit. The analysis unit reads internal communications and analyzes words and surrounding context. For example, the analysis unit analyzes the content of employee statements and checks whether specific words or phrases are included. The analysis unit can analyze the context of statement content using natural language processing technology. For example, the analysis unit inputs the content of employee statements as text data and analyzes the context using natural language processing technology. The detection unit detects statements with potential risk based on the content analyzed by the analysis unit and accumulates them in a database. For example, the detection unit detects aggressive statements and accumulates them in the database. The detection unit can evaluate the risk of statement content using AI. For example, the detection unit detects statements containing aggressive words or discriminatory expressions and accumulates them in the database. The diagnosis unit performs intellectual personality diagnosis based on the data accumulated by the detection unit and categorizes employees. For example, the diagnosis unit analyzes the content and tone of employee statements and diagnoses the personality and intellectual characteristics of the employee. The diagnosis unit can evaluate the intellectual personality of statement content using AI. For example, the diagnosis unit inputs the content of employee statements as text data and diagnoses intellectual personality using AI. As a result, the system can automate the analysis of internal communications, detection of risk, and intellectual personality diagnosis, thereby realizing the prevention of harassment and aggregation of human resource information. Specifically, the system is equipped with natural language processing models such as Transformer-based large language models, bidirectional LSTM, and convolutional neural networks as the analysis unit, and a data acquisition module that collects various internal communication data such as internal chat, email, and voice conversation logs. The data acquisition module accepts input such as text data (e.g., UTF-8 encoded string arrays, up to 4096 tokens per sample), audio data (e.g., WAV format sampled at 16 kHz, up to 60 seconds per utterance), and image data (e.g., PNG format screenshots, 1280×720 pixels per image). These data are preprocessed by a preprocessing unit for noise removal, tokenization, speech-to-text conversion (ASR), removal of unnecessary words, and normalization. The analysis unit extracts word embedding vectors (e.g., 768-dimensional vectors of BERT) and contextual features, and handles input examples such as text data in which a supervisor says to a subordinate, “You are useless,” and utterance records extracted from audio data during meetings. The analysis unit outputs an aggressive word list (e.g., ‘useless’, ‘incompetent’, ‘fool’) and context-dependent risk scores (e.g., 0.85/1.0) from these inputs. The detection unit receives the output of the analysis unit and performs threshold judgment (e.g., detection when the risk score is 0.7 or higher) and rule-based filtering (e.g., only statements between specific positions), and records the detection results (e.g., risk statement label, speaker ID, timestamp, risk score) as structured data in the database. Examples of output from the detection unit include “Statement ID: 12345, Risk Statement: True, Score: 0.92, Speaker: Employee A, Date: 2024-06-01 10:15.” The diagnosis unit inputs the statement history and tone information accumulated by the detection unit as time-series vectors (e.g., emotion score arrays for each statement, length 30), and uses a personality diagnosis model (e.g., multilayer perceptron for Big Five personality trait estimation, output: [Extraversion: 0.7, Agreeableness: 0.4, Conscientiousness: 0.8, Neuroticism: 0.2, Openness: 0.6]) to classify employees into multiple intellectual personality categories. Examples of output from the diagnosis unit include “Employee A: High Extraversion, Low Agreeableness, Intellectual Trait: Logical Type.” These outputs are automatically linked to human resource management systems and harassment prevention dashboards and are utilized for early intervention by administrators and planning of organizational improvement measures. Unlike conventional human visual audits and questionnaire tabulation, this system uses computer-specific unconventional methods such as automatic analysis in high-dimensional feature space, hybrid judgment of rule-based and machine learning, and automatic detection of time-series changes, thereby achieving significant improvements in processing speed, reduction of false detection rate, and efficiency in data management. Specific application fields include corporate harassment prevention, appropriate allocation of human resources, healthy internal communication, risk management, and employee mental health support.

The analysis unit can analyze the content of employee statements and check whether specific words or phrases are included. For example, the analysis unit analyzes the content of employee statements and checks whether specific words or phrases are included. The analysis unit can analyze the context of statement content using natural language processing technology. For example, the analysis unit inputs the content of employee statements as text data and analyzes the context using natural language processing technology. As a result, by analyzing the content of employee statements in detail and checking for specific words or phrases, it is possible to detect statements with potential risk at an early stage. Specifically, the analysis unit uses natural language processing models such as Transformer-based large language models, bidirectional LSTM, and convolutional neural networks to tokenize the input text data (e.g., UTF-8 encoded string arrays, up to 4096 tokens per sample) and extract word embedding vectors (e.g., 768-dimensional vectors of BERT) and contextual features. Examples of input include text data in which a supervisor says to a subordinate, “You are useless,” and utterance records extracted from audio data during meetings. The analysis unit outputs an aggressive word list (e.g., ‘useless’, ‘incompetent’, ‘fool’) and context-dependent risk scores (e.g., 0.85/1.0) from these inputs. The analysis unit performs not only simple word matching but also context-dependent semantic analysis and high-dimensional feature extraction considering the relationship before and after the statement, thereby achieving early detection of risk statements with higher accuracy than conventional rule-based methods. As a result, the analysis unit contributes to maintaining the soundness of internal communications and preventing harassment and improving the efficiency of human resource management. Application fields include corporate harassment prevention, organizational risk management, and employee mental health support.

The detection unit can detect specific aggressive statements and accumulate them in the database. For example, the detection unit detects specific aggressive statements and accumulates them in the database. The detection unit can evaluate the risk of statement content using AI. For example, the detection unit detects statements containing aggressive words or discriminatory expressions and accumulates them in the database. As a result, by detecting and accumulating aggressive statements in the database, it is possible to prevent harassment in advance. Specifically, the detection unit receives the aggressive word list and risk scores (e.g., 0.85/1.0) output from the analysis unit and performs threshold judgment (e.g., detection when the risk score is 0.7 or higher) and rule-based filtering (e.g., only statements between specific positions). The detection unit records the detection results (e.g., risk statement label, speaker ID, timestamp, risk score) as structured data in the database. Examples of output from the detection unit include “Statement ID: 12345, Risk Statement: True, Score: 0.92, Speaker: Employee A, Date: 2024-06-01 10:15.” The detection unit uses AI models such as multilayer perceptron, decision tree, and SVM to score the risk from the features of statement content and automatically record and notify when the threshold is exceeded. Unlike conventional human visual audits and simple keyword detection, the detection unit performs automatic judgment in high-dimensional feature space and automatic detection of time-series changes, thereby achieving technical effects such as reduction of false detection rate and improvement of processing speed. Application fields include corporate harassment prevention, risk management, and early detection of organizational troubles.

The diagnosis unit can analyze the content or tone of employee statements and diagnose the personality or intellectual characteristics of the employee. For example, the diagnosis unit analyzes the content or tone of employee statements and diagnoses the personality or intellectual characteristics of the employee. The diagnosis unit can evaluate the intellectual personality of statement content using AI. For example, the diagnosis unit inputs the content of employee statements as text data and diagnoses intellectual personality using AI. As a result, by analyzing the content and tone of employee statements, it is possible to diagnose personality and intellectual characteristics and appropriately categorize employees. Specifically, the diagnosis unit inputs the statement history and tone information accumulated by the detection unit as time-series vectors (e.g., emotion score arrays for each statement, length 30), and uses a personality diagnosis model (e.g., multilayer perceptron for Big Five personality trait estimation, output: [Extraversion: 0.7, Agreeableness: 0.4, Conscientiousness: 0.8, Neuroticism: 0.2, Openness: 0.6]) to classify employees into multiple intellectual personality categories. Examples of output from the diagnosis unit include “Employee A: High Extraversion, Low Agreeableness, Intellectual Trait: Logical Type.” The diagnosis unit uses AI models such as multilayer perceptron, random forest, and SVM to analyze features of statement content and tone changes in high-dimensional space and quantitatively evaluate the personality and intellectual characteristics of employees. The output of the diagnosis unit is automatically linked to human resource management systems and harassment prevention dashboards and is utilized for early intervention by administrators and planning of organizational improvement measures. Unlike conventional questionnaires and visual evaluations, the diagnosis unit integrally analyzes time-series data and multiple features, thereby achieving technical effects such as improvement of diagnosis accuracy and optimal allocation of human resources. Application fields include human resource management, healthy internal communication, and employee mental health support.

The analysis unit can estimate the user's emotion and adjust the accuracy of the analysis based on the estimated emotion of the user. For example, the analysis unit estimates the user's emotion and adjusts the accuracy of the analysis based on the estimated emotion. The analysis unit is realized by using an emotion estimation function with an emotion engine or generative AI. The generative AI may be a text generation AI (e.g., LLM) or a multimodal generative AI, but is not limited to these examples. For example, when the user is feeling stressed, the analysis unit increases the accuracy of the analysis and performs more detailed analysis. When the user is relaxed, the analysis unit adjusts the accuracy and performs only the minimum necessary analysis. Furthermore, when the user is excited, the analysis unit adjusts the accuracy and performs analysis considering emotional fluctuations. By adjusting the accuracy of the analysis based on the user's emotion, more appropriate analysis becomes possible. Specifically, the analysis unit accepts input such as user statement text data (e.g., UTF-8 encoded string arrays, up to 4096 tokens per sample), audio data (e.g., WAV format sampled at 16 kHz, up to 60 seconds per utterance), and image data (e.g., PNG format screenshots, 1280×720 pixels per image). The analysis unit performs preprocessing such as noise removal, tokenization, speech-to-text conversion (ASR), removal of unnecessary words, and normalization on these data in the preprocessing unit. As the emotion estimation function, the analysis unit uses a combination of Transformer-based large language models, bidirectional LSTM, convolutional neural networks, or speech emotion recognition models (e.g., acoustic feature extraction+RNN). Examples of input to the analysis unit include text data in which the user says “I can't take it anymore” and audio data in which the voice is trembling during a meeting. The analysis unit outputs emotion categories (e.g., stress, relaxation, excitement), emotion intensity scores (e.g., stress 0.85/1.0), and emotion fluctuation patterns (e.g., increasing stress trend over the past 10 statements) from these inputs. The analysis unit dynamically adjusts analysis accuracy parameters (e.g., threshold, depth of feature extraction, number of model inferences) according to the estimated emotion score. For example, when the stress score is high, the context analysis window width is widened and detailed feature extraction is performed. In a relaxed state, the analysis unit switches to a simplified analysis mode with reduced computational load. In an excited state, the analysis unit strengthens time-series analysis considering emotional fluctuations. The analysis unit optimizes the detection accuracy of risk statements and the reliability of personality diagnosis based on these adjustment results. Examples of output from the analysis unit include “Statement ID: 56789, Emotion: Stress, Score: 0.92, Analysis Accuracy: High” and “Statement ID: 56790, Emotion: Relaxation, Score: 0.15, Analysis Accuracy: Standard.” The analysis unit links these outputs to subsequent detection units and diagnosis units to improve the overall system accuracy. Unlike conventional subjective emotion judgment by humans and uniform analysis processing, the analysis unit automatically performs emotion estimation and dynamic optimization of analysis accuracy parameters in high-dimensional feature space, thereby achieving technical effects such as improvement of analysis accuracy, efficient use of computational resources, and reduction of false detection rate. Application fields include corporate harassment prevention, employee mental health support, customer support emotion analysis, and risk management.

The analysis unit can adjust the analysis algorithm by referring to the employee's past statement history during analysis. For example, the analysis unit adjusts the analysis algorithm by referring to the employee's past statement history during analysis. The analysis unit can optimize the analysis algorithm based on past statement history using AI. For example, the analysis unit detects specific patterns based on the employee's past statement history and optimizes the analysis algorithm. The analysis unit can also extract frequently used words or phrases from the employee's past statement history and reflect them in the analysis algorithm. Furthermore, the analysis unit can analyze the employee's past statement history and adjust the analysis algorithm considering specific tone or emotional changes. By referring to the employee's past statement history, the analysis algorithm can be optimized and the accuracy of the analysis can be improved. Specifically, the analysis unit refers to a statement history database for each employee (e.g., structured data including statement ID, statement text, timestamp, emotion score, tone information). The analysis unit extracts the statement content of the past 30 days as time-series vectors (e.g., feature arrays for each statement, length 30) and inputs them into natural language processing models (e.g., Transformer-based large language models or bidirectional LSTM). The analysis unit automatically extracts frequently used word lists (e.g., [‘deadline’, ‘delay’, ‘urgent’]), phrase patterns (e.g., ‘urgent response’, ‘reconfirmation’), and tone changes (e.g., transition from positive to negative). Based on these features, the analysis unit individually optimizes parameters of the analysis algorithm (e.g., threshold for risk statement detection, context window width, weights of emotion estimation models). For example, for employees with many aggressive statements in the past, the threshold for risk score is set lower and detailed analysis is performed. Conversely, for employees with few problematic statements in the past, a standard analysis mode is applied. The analysis unit uses time-series analysis algorithms (e.g., autoregressive models or LSTM) to detect change points or anomalies in statement patterns and dynamically switch the analysis algorithm. Examples of output from the analysis unit include “Employee B: 5 aggressive statements in the past 30 days, risk threshold 0.6, analysis mode: detailed” and “Employee C: stable statement tone, threshold 0.8, analysis mode: standard.” The analysis unit links these outputs to the detection unit and diagnosis unit to improve the overall system accuracy. Unlike conventional uniform analysis processing and human reference to history, the analysis unit automatically performs history analysis and algorithm optimization in high-dimensional feature space, thereby achieving technical effects such as improvement of analysis accuracy, reduction of false detection rate, and flexible response by individual optimization. Application fields include corporate harassment prevention, employee behavior analysis, risk management, and human resource management.

The analysis unit can apply different analysis methods according to the employee's position or department during analysis. For example, the analysis unit applies different analysis methods according to the employee's position or department during analysis. The analysis unit can apply analysis methods according to position or department using AI. For example, the analysis unit distinguishes statements between supervisors and subordinates according to the employee's position and applies appropriate analysis methods. The analysis unit can also distinguish statement content between technical departments and sales departments according to the employee's department and apply appropriate analysis methods. Furthermore, the analysis unit can prioritize analysis of statements related to specific business content according to the employee's position or department. By applying analysis methods according to the employee's position or department, more appropriate analysis becomes possible. Specifically, the analysis unit refers to an employee information database (e.g., structured data including employee ID, position, department, business content) and performs analysis processing linked to statement data. The analysis unit extracts position information (e.g., manager, general staff, leader) and department information (e.g., technical department, sales department, administration department) as features and reflects them in the selection of analysis algorithms and parameter settings. For example, for statements from supervisors to subordinates, a risk statement detection model considering power balance (e.g., SVM with inter-position weighting) is applied. For statements in technical departments, a context analysis model using a technical term dictionary (e.g., custom BERT) is used. For statements in sales departments, a phrase detection algorithm specific to customer response (e.g., N-gram based) is applied. The analysis unit prioritizes extraction and detailed analysis of statements related to business content (e.g., project progress, deadline adjustment). Examples of output from the analysis unit include “Statement ID: 78901, Position: Manager, Department: Sales, Analysis Model: Sales-specialized, Risk Score: 0.75” and “Statement ID: 78902, Position: General Staff, Department: Technical, Analysis Model: Technical Term Enhanced, Risk Score: 0.45.” The analysis unit links these outputs to the detection unit and diagnosis unit to improve the overall system accuracy. Unlike conventional uniform analysis processing and human consideration of position and department, the analysis unit automatically utilizes position and department information in high-dimensional feature space and dynamically switches analysis methods, thereby achieving technical effects such as improvement of analysis accuracy, reduction of false detection rate, and flexible response to business characteristics. Application fields include corporate harassment prevention, organizational risk management, human resource management, and business process optimization.

The analysis unit can estimate the user's emotion and adjust the display method of the analysis result based on the estimated emotion of the user. For example, the analysis unit estimates the user's emotion and adjusts the display method of the analysis result based on the estimated emotion. The analysis unit is realized by using an emotion estimation function with an emotion engine or generative AI. The generative AI may be a text generation AI (e.g., LLM) or a multimodal generative AI, but is not limited to these examples. For example, when the user is feeling stressed, the analysis unit provides a simple and highly visible display method. When the user is relaxed, the analysis unit can provide a display method including detailed information. Furthermore, when the user is excited, the analysis unit can provide a visually stimulating display method. By adjusting the display method of the analysis result based on the user's emotion, a display that is easy for the user to see becomes possible. Specifically, the analysis unit accepts user statement text and audio data as input and uses emotion estimation models (e.g., Transformer-based large language models or speech emotion recognition models) to output emotion categories (e.g., stress, relaxation, excitement) and emotion intensity scores (e.g., 0.85/1.0). The analysis unit dynamically adjusts display parameters such as UI layout, color scheme, amount of information, and graph type of dashboards and notification screens according to the estimated emotion. For example, in a stress state, only important information is emphasized and the color tone is set to a calm one. In a relaxed state, detailed analysis results and history graphs are displayed. In an excited state, interactive displays using animation and accent colors are provided. Examples of output from the analysis unit include “User ID: 123, Emotion: Stress, Display Mode: Simple” and “User ID: 124, Emotion: Relaxation, Display Mode: Detailed.” The analysis unit links these outputs to the frontend display module to optimize the user experience. Unlike conventional uniform information display and manual adjustment by humans, the analysis unit automatically optimizes emotion estimation and display parameters, thereby achieving technical effects such as improvement of usability, reduction of stress due to information overload, and realization of situation-adaptive UI. Application fields include corporate harassment prevention dashboards, employee mental health support systems, and customer support analysis tools.

The analysis unit can perform analysis based on the employee's geographic location information during analysis. For example, the analysis unit performs analysis based on the employee's geographic location information during analysis. The analysis unit can perform analysis considering geographic location information using AI. For example, the analysis unit prioritizes analysis of statements related to specific regions based on the employee's geographic location information. The analysis unit can also perform analysis reflecting regional characteristics by considering the employee's geographic location information. Furthermore, the analysis unit can analyze statement patterns in specific regions based on the employee's geographic location information. By considering the employee's geographic location information, analysis reflecting regional characteristics becomes possible. Specifically, the analysis unit acquires employee location information data (e.g., structured data including GPS coordinates, office ID, city name) linked to statement data. The analysis unit extracts geographic features (e.g., city, country, office division) and reflects them in the parameters and model selection of the analysis algorithm. For example, the analysis unit detects statement patterns frequently occurring in specific regions (e.g., dialects or local expressions in the Kansai office) and applies region-specialized natural language processing models (e.g., BERT with regional language dictionary). The analysis unit automatically aggregates regional risk trends (e.g., increase in stress statements in urban areas, increase in cooperative statements in local offices) and reflects them in the analysis results. Examples of output from the analysis unit include “Statement ID: 34567, Location: Tokyo Headquarters, Analysis Model: Urban Type, Risk Score: 0.65” and “Statement ID: 34568, Location: Osaka Branch, Analysis Model: Kansai Specialized, Risk Score: 0.72.” The analysis unit links these outputs to the detection unit and diagnosis unit and utilizes them for planning organizational improvement measures considering regional characteristics. Unlike conventional uniform analysis processing and human consideration of regional characteristics, the analysis unit automatically utilizes geographic location information in high-dimensional feature space and dynamically switches region-specialized models, thereby achieving technical effects such as improvement of analysis accuracy, early detection of regional risks, and cross-organizational data utilization. Application fields include corporate multi-site harassment prevention, global human resource management, and region-specific risk management.

The analysis unit can analyze the employee's SNS activity during analysis and analyze related statements. For example, the analysis unit analyzes the employee's SNS activity during analysis and analyzes related statements. The analysis unit can perform analysis based on SNS activity using AI. For example, the analysis unit analyzes statements related to specific topics based on the employee's SNS activity. The analysis unit can also analyze changes in tone or emotion by analyzing the employee's SNS activity. Furthermore, the analysis unit can analyze specific keywords or hashtags based on the employee's SNS activity. By analyzing the employee's SNS activity, it is possible to analyze related statements and perform more detailed analysis. Specifically, the analysis unit collects employee SNS post data (e.g., structured data including post ID, post text, timestamp, hashtag, emotion score) and integrates it with statement data for analysis processing. The analysis unit automatically performs topic classification on SNS (e.g., topic estimation by LDA), keyword extraction (e.g., TF-IDF score), hashtag frequency analysis, and tone change (e.g., positive/negative judgment). The analysis unit performs correlation analysis between SNS activity and internal statements (e.g., tendency for aggressive internal statements when negative posts increase on SNS) and dynamically adjusts parameters of the analysis algorithm (e.g., threshold for risk statement detection, weights of emotion estimation models). Examples of output from the analysis unit include “Employee D: SNS Topic: Deadline Delay, Internal Statement: Aggressive, Risk Score: 0.82” and “Employee E: SNS Hashtag: #WorkStyleReform, Internal Statement: Cooperative, Risk Score: 0.35.” The analysis unit links these outputs to the detection unit and diagnosis unit and utilizes them for organizational risk analysis and human resource management reflecting SNS activity. Unlike conventional analysis targeting only internal data and human monitoring of SNS, the analysis unit automatically integrates SNS activity in high-dimensional feature space and dynamically optimizes the analysis algorithm, thereby achieving technical effects such as improvement of analysis accuracy, early detection of risks, and cross-organizational data utilization. Application fields include corporate harassment prevention, SNS risk management, human resource management, and brand image protection.

The detection unit can estimate the user's emotion and adjust the detection criteria based on the estimated emotion of the user. For example, the detection unit estimates the user's emotion and adjusts the detection criteria based on the estimated emotion. The detection unit is realized by using an emotion estimation function with an emotion engine or generative AI. The generative AI may be a text generation AI (e.g., LLM) or a multimodal generative AI, but is not limited to these examples. For example, when the user is feeling stressed, the detection unit tightens the detection criteria and performs more detailed detection. When the user is relaxed, the detection unit loosens the detection criteria and performs only the minimum necessary detection. Furthermore, when the user is excited, the detection unit adjusts the detection criteria and performs detection considering emotional fluctuations. By adjusting the detection criteria based on the user's emotion, more appropriate detection becomes possible. Specifically, the detection unit accepts input such as user statement text data (e.g., UTF-8 encoded string arrays, up to 4096 tokens per sample), audio data (e.g., WAV format sampled at 16 kHz, up to 60 seconds per utterance), and image data (e.g., PNG format screenshots, 1280×720 pixels per image). The detection unit performs preprocessing such as noise removal, tokenization, speech-to-text conversion (ASR), removal of unnecessary words, and normalization on these data in the preprocessing unit. As the emotion estimation function, the detection unit uses a combination of Transformer-based large language models, bidirectional LSTM, convolutional neural networks, and speech emotion recognition models (e.g., acoustic feature extraction+RNN). Examples of input to the detection unit include text data in which the user says “I can't take it anymore” and audio data in which the voice is trembling during a meeting. The detection unit outputs emotion categories (e.g., stress, relaxation, excitement), emotion intensity scores (e.g., stress 0.85/1.0), and emotion fluctuation patterns (e.g., increasing stress trend over the past 10 statements) from these inputs. The detection unit dynamically adjusts detection criteria parameters (e.g., threshold for risk statement detection, depth of feature extraction, number of model inferences) according to the estimated emotion score. For example, when the stress score is high, the threshold for risk statement detection is lowered and detailed feature extraction is performed. In a relaxed state, the detection unit switches to a simplified detection mode with reduced computational load. In an excited state, the detection unit strengthens time-series detection considering emotional fluctuations. The detection unit optimizes the detection accuracy of risk statements and notification frequency based on these adjustment results. Examples of output from the detection unit include “Statement ID: 56789, Emotion: Stress, Detection Criteria: Strict” and “Statement ID: 56790, Emotion: Relaxation, Detection Criteria: Standard.” The detection unit links these outputs to subsequent database recording and administrator notification modules to improve the overall system accuracy. Unlike conventional subjective emotion judgment by humans and uniform detection processing, the detection unit automatically performs emotion estimation and dynamic optimization of detection criteria parameters in high-dimensional feature space, thereby achieving technical effects such as improvement of detection accuracy, efficient use of computational resources, and reduction of false detection rate. Application fields include corporate harassment prevention, employee mental health support, customer support emotion analysis, and risk management.

The detection unit can adjust the detection algorithm by referring to the employee's past statement history during detection. For example, the detection unit adjusts the detection algorithm by referring to the employee's past statement history during detection. The detection unit can optimize the detection algorithm based on past statement history using AI. For example, the detection unit detects specific patterns based on the employee's past statement history and optimizes the detection algorithm. The detection unit can also extract frequently used words or phrases from the employee's past statement history and reflect them in the detection algorithm. Furthermore, the detection unit can analyze the employee's past statement history and adjust the detection algorithm considering specific tone or emotional changes. By referring to the employee's past statement history, the detection algorithm can be optimized and the accuracy of detection can be improved. Specifically, the detection unit refers to a statement history database for each employee (e.g., structured data including statement ID, statement text, timestamp, emotion score, tone information). The detection unit extracts the statement content of the past 30 days as time-series vectors (e.g., feature arrays for each statement, length 30) and inputs them into natural language processing models (e.g., Transformer-based large language models or bidirectional LSTM). The detection unit automatically extracts frequently used word lists (e.g., [‘deadline’, ‘delay’, ‘urgent’]), phrase patterns (e.g., ‘urgent response’, ‘reconfirmation’), and tone changes (e.g., transition from positive to negative). Based on these features, the detection unit individually optimizes parameters of the detection algorithm (e.g., threshold for risk statement detection, context window width, weights of emotion estimation models). For example, for employees with many aggressive statements in the past, the threshold for risk score is set lower and detailed detection is performed. Conversely, for employees with few problematic statements in the past, a standard detection mode is applied. The detection unit uses time-series analysis algorithms (e.g., autoregressive models or LSTM) to detect change points or anomalies in statement patterns and dynamically switch the detection algorithm. Examples of output from the detection unit include “Employee B: 5 aggressive statements in the past 30 days, risk threshold 0.6, detection mode: detailed” and “Employee C: stable statement tone, threshold 0.8, detection mode: standard.” The detection unit links these outputs to database recording and administrator notification modules to improve the overall system accuracy. Unlike conventional uniform detection processing and human reference to history, the detection unit automatically performs history analysis and algorithm optimization in high-dimensional feature space, thereby achieving technical effects such as improvement of detection accuracy, reduction of false detection rate, and flexible response by individual optimization. Application fields include corporate harassment prevention, employee behavior analysis, risk management, and human resource management.

The detection unit can apply different detection methods according to the employee's position or department during detection. For example, the detection unit applies different detection methods according to the employee's position or department during detection. The detection unit can apply detection methods according to position or department using AI. For example, the detection unit distinguishes statements between supervisors and subordinates according to the employee's position and applies appropriate detection methods. The detection unit can also distinguish statement content between technical departments and sales departments according to the employee's department and apply appropriate detection methods. Furthermore, the detection unit can prioritize detection of statements related to specific business content according to the employee's position or department. By applying detection methods according to the employee's position or department, more appropriate detection becomes possible. Specifically, the detection unit refers to an employee information database (e.g., structured data including employee ID, position, department, business content) and performs detection processing linked to statement data. The detection unit extracts position information (e.g., manager, general staff, leader) and department information (e.g., technical department, sales department, administration department) as features and reflects them in the selection of detection algorithms and parameter settings. For example, for statements from supervisors to subordinates, a risk statement detection model considering power balance (e.g., SVM with inter-position weighting) is applied. For statements in technical departments, a context analysis model using a technical term dictionary (e.g., custom BERT) is used. For statements in sales departments, a phrase detection algorithm specific to customer response (e.g., N-gram based) is applied. The detection unit prioritizes extraction and detailed detection of statements related to business content (e.g., project progress, deadline adjustment). Examples of output from the detection unit include “Statement ID: 78901, Position: Manager, Department: Sales, Detection Model: Sales-specialized, Risk Score: 0.75” and “Statement ID: 78902, Position: General Staff, Department: Technical, Detection Model: Technical Term Enhanced, Risk Score: 0.45.” The detection unit links these outputs to database recording and administrator notification modules to improve the overall system accuracy. Unlike conventional uniform detection processing and human consideration of position and department, the detection unit automatically utilizes position and department information in high-dimensional feature space and dynamically switches detection methods, thereby achieving technical effects such as improvement of detection accuracy, reduction of false detection rate, and flexible response to business characteristics. Application fields include corporate harassment prevention, organizational risk management, human resource management, and business process optimization.

The detection unit can estimate the user's emotion and adjust the display method of the detection result based on the estimated emotion of the user. For example, the detection unit estimates the user's emotion and adjusts the display method of the detection result based on the estimated emotion. The detection unit is realized by using an emotion estimation function with an emotion engine or generative AI. The generative AI may be a text generation AI (e.g., LLM) or a multimodal generative AI, but is not limited to these examples. For example, when the user is feeling stressed, the detection unit provides a simple and highly visible display method. When the user is relaxed, the detection unit can provide a display method including detailed information. Furthermore, when the user is excited, the detection unit can provide a visually stimulating display method. By adjusting the display method of the detection result based on the user's emotion, a display that is easy for the user to see becomes possible. Specifically, the detection unit accepts user statement text and audio data as input and uses emotion estimation models (e.g., Transformer-based large language models or speech emotion recognition models) to output emotion categories (e.g., stress, relaxation, excitement) and emotion intensity scores (e.g., 0.85/1.0). The detection unit dynamically adjusts display parameters such as UI layout, color scheme, amount of information, and graph type of dashboards and notification screens according to the estimated emotion. For example, in a stress state, only important information is emphasized and the color tone is set to a calm one. In a relaxed state, detailed detection results and history graphs are displayed. In an excited state, interactive displays using animation and accent colors are provided. Examples of output from the detection unit include “User ID: 123, Emotion: Stress, Display Mode: Simple” and “User ID: 124, Emotion: Relaxation, Display Mode: Detailed.” The detection unit links these outputs to the frontend display module to optimize the user experience. Unlike conventional uniform information display and manual adjustment by humans, the detection unit automatically optimizes emotion estimation and display parameters, thereby achieving technical effects such as improvement of usability, reduction of stress due to information overload, and realization of situation-adaptive UI. Application fields include corporate harassment prevention dashboards, employee mental health support systems, and customer support analysis tools.

The detection unit can perform detection based on the employee's geographic location information during detection. For example, the detection unit performs detection based on the employee's geographic location information during detection. The detection unit can perform detection considering geographic location information using AI. For example, the detection unit prioritizes detection of statements related to specific regions based on the employee's geographic location information. The detection unit can also perform detection reflecting regional characteristics by considering the employee's geographic location information. Furthermore, the detection unit can detect statement patterns in specific regions based on the employee's geographic location information. By considering the employee's geographic location information, detection reflecting regional characteristics becomes possible. Specifically, the detection unit acquires employee location information data (e.g., structured data including GPS coordinates, office ID, city name) linked to statement data. The detection unit extracts geographic features (e.g., city, country, office division) and reflects them in the parameters and model selection of the detection algorithm. For example, the detection unit detects statement patterns frequently occurring in specific regions (e.g., dialects or local expressions in the Kansai office) and applies region-specialized natural language processing models (e.g., BERT with regional language dictionary). The detection unit automatically aggregates regional risk trends (e.g., increase in stress statements in urban areas, increase in cooperative statements in local offices) and reflects them in the detection results. Examples of output from the detection unit include “Statement ID: 34567, Location: Tokyo Headquarters, Detection Model: Urban Type, Risk Score: 0.65” and “Statement ID: 34568, Location: Osaka Branch, Detection Model: Kansai Specialized, Risk Score: 0.72.” The detection unit links these outputs to database recording and administrator notification modules and utilizes them for planning organizational improvement measures considering regional characteristics. Unlike conventional uniform detection processing and human consideration of regional characteristics, the detection unit automatically utilizes geographic location information in high-dimensional feature space and dynamically switches region-specialized models, thereby achieving technical effects such as improvement of detection accuracy, early detection of regional risks, and cross-organizational data utilization. Application fields include corporate multi-site harassment prevention, global human resource management, and region-specific risk management.

The detection unit can analyze the employee's SNS activity during detection and detect related statements. For example, the detection unit analyzes the employee's SNS activity during detection and detects related statements. The detection unit can perform detection based on SNS activity using AI. For example, the detection unit detects statements related to specific topics based on the employee's SNS activity. The detection unit can also detect changes in tone or emotion by analyzing the employee's SNS activity. Furthermore, the detection unit can detect specific keywords or hashtags based on the employee's SNS activity. By analyzing the employee's SNS activity, it is possible to detect related statements and perform more detailed detection. Specifically, the detection unit collects employee SNS post data (e.g., structured data including post ID, post text, timestamp, hashtag, emotion score) and integrates it with statement data for detection processing. The detection unit automatically performs topic classification on SNS (e.g., topic estimation by LDA), keyword extraction (e.g., TF-IDF score), hashtag frequency analysis, and tone change (e.g., positive/negative judgment). The detection unit performs correlation analysis between SNS activity and internal statements (e.g., tendency for aggressive internal statements when negative posts increase on SNS) and dynamically adjusts parameters of the detection algorithm (e.g., threshold for risk statement detection, weights of emotion estimation models). Examples of output from the detection unit include “Employee D: SNS Topic: Deadline Delay, Internal Statement: Aggressive, Risk Score: 0.82” and “Employee E: SNS Hashtag: #WorkStyleReform, Internal Statement: Cooperative, Risk Score: 0.35.” The detection unit links these outputs to database recording and administrator notification modules and utilizes them for organizational risk analysis and human resource management reflecting SNS activity. Unlike conventional detection targeting only internal data and human monitoring of SNS, the detection unit automatically integrates SNS activity in high-dimensional feature space and dynamically optimizes the detection algorithm, thereby achieving technical effects such as improvement of detection accuracy, early detection of risks, and cross-organizational data utilization. Application fields include corporate harassment prevention, SNS risk management, human resource management, and brand image protection.

The diagnosis unit can estimate the user's emotion and adjust the accuracy of the diagnosis based on the estimated emotion of the user. For example, the diagnosis unit estimates the user's emotion and adjusts the accuracy of the diagnosis based on the estimated emotion. The diagnosis unit is realized by using an emotion estimation function with an emotion engine or generative AI. The generative AI may be a text generation AI (e.g., LLM) or a multimodal generative AI, but is not limited to these examples. For example, when the user is feeling stressed, the diagnosis unit increases the accuracy of the diagnosis and performs more detailed diagnosis. When the user is relaxed, the diagnosis unit adjusts the accuracy and performs only the minimum necessary diagnosis. Furthermore, when the user is excited, the diagnosis unit adjusts the accuracy and performs diagnosis considering emotional fluctuations. By adjusting the accuracy of the diagnosis based on the user's emotion, more appropriate diagnosis becomes possible. Specifically, the diagnosis unit accepts input such as user statement text data (e.g., UTF-8 encoded string arrays, up to 4096 tokens per sample), audio data (e.g., WAV format sampled at 16 kHz, up to 60 seconds per utterance), and image data (e.g., PNG format screenshots, 1280×720 pixels per image). The diagnosis unit performs preprocessing such as noise removal, tokenization, speech-to-text conversion (ASR), removal of unnecessary words, and normalization on these data in the preprocessing unit. As the emotion estimation function, the diagnosis unit uses a combination of Transformer-based large language models, bidirectional LSTM, convolutional neural networks, and speech emotion recognition models (e.g., acoustic feature extraction+RNN). Examples of input to the diagnosis unit include text data in which the user says “I can't take it anymore” and audio data in which the voice is trembling during a meeting. The diagnosis unit outputs emotion categories (e.g., stress, relaxation, excitement), emotion intensity scores (e.g., stress 0.85/1.0), and emotion fluctuation patterns (e.g., increasing stress trend over the past 10 statements) from these inputs. The diagnosis unit dynamically adjusts diagnosis accuracy parameters (e.g., depth of feature extraction, number of model inferences, detail level of diagnosis items) according to the estimated emotion score. For example, when the stress score is high, the number of layers in the personality diagnosis model is increased and detailed feature extraction is performed. In a relaxed state, the diagnosis unit switches to a simplified diagnosis mode with reduced computational load. In an excited state, the diagnosis unit strengthens time-series diagnosis considering emotional fluctuations. The diagnosis unit optimizes the reliability of personality diagnosis and the accuracy of category classification based on these adjustment results. Examples of output from the diagnosis unit include “User ID: 56789, Emotion: Stress, Diagnosis Accuracy: High, Diagnosis Result: Logical Type” and “User ID: 56790, Emotion: Relaxation, Diagnosis Accuracy: Standard, Diagnosis Result: Cooperative Type.” The diagnosis unit links these outputs to human resource management systems and harassment prevention dashboards and utilizes them for early intervention by administrators and planning of organizational improvement measures. Unlike conventional subjective emotion judgment by humans and uniform diagnosis processing, the diagnosis unit automatically performs emotion estimation and dynamic optimization of diagnosis accuracy parameters in high-dimensional feature space, thereby achieving technical effects such as improvement of diagnosis accuracy, efficient use of computational resources, and reduction of misdiagnosis rate. Application fields include corporate human resource management, employee mental health support, organizational communication analysis, and risk management.

The diagnosis unit can adjust the diagnosis algorithm by referring to the employee's past statement history during diagnosis. For example, the diagnosis unit adjusts the diagnosis algorithm by referring to the employee's past statement history during diagnosis. The diagnosis unit can optimize the diagnosis algorithm based on past statement history using AI. For example, the diagnosis unit detects specific patterns based on the employee's past statement history and optimizes the diagnosis algorithm. The diagnosis unit can also extract frequently used words or phrases from the employee's past statement history and reflect them in the diagnosis algorithm. Furthermore, the diagnosis unit can analyze the employee's past statement history and adjust the diagnosis algorithm considering specific tone or emotional changes. By referring to the employee's past statement history, the diagnosis algorithm can be optimized and the accuracy of diagnosis can be improved. Specifically, the diagnosis unit refers to a statement history database for each employee (e.g., structured data including statement ID, statement text, timestamp, emotion score, tone information). The diagnosis unit extracts the statement content of the past 30 days as time-series vectors (e.g., feature arrays for each statement, length 30) and inputs them into natural language processing models (e.g., Transformer-based large language models or bidirectional LSTM). The diagnosis unit automatically extracts frequently used word lists (e.g., [‘deadline’, ‘delay’, ‘urgent’]), phrase patterns (e.g., ‘urgent response’, ‘reconfirmation’), and tone changes (e.g., transition from positive to negative). Based on these features, the diagnosis unit individually optimizes parameters of the diagnosis algorithm (e.g., weights of diagnosis items, context window width, weights of emotion estimation models). For example, for employees with many aggressive statements in the past, the weights of diagnosis items are adjusted and detailed diagnosis is performed. Conversely, for employees with few problematic statements in the past, a standard diagnosis mode is applied. The diagnosis unit uses time-series analysis algorithms (e.g., autoregressive models or LSTM) to detect change points or anomalies in statement patterns and dynamically switch the diagnosis algorithm. Examples of output from the diagnosis unit include “Employee B: 5 aggressive statements in the past 30 days, diagnosis weight: high, diagnosis mode: detailed” and “Employee C: stable statement tone, diagnosis weight: standard, diagnosis mode: standard.” The diagnosis unit links these outputs to human resource management systems and harassment prevention dashboards to improve the overall system accuracy. Unlike conventional uniform diagnosis processing and human reference to history, the diagnosis unit automatically performs history analysis and algorithm optimization in high-dimensional feature space, thereby achieving technical effects such as improvement of diagnosis accuracy, reduction of misdiagnosis rate, and flexible response by individual optimization. Application fields include corporate harassment prevention, employee behavior analysis, risk management, and human resource management.

The diagnosis unit can apply different diagnosis methods according to the employee's position or department during diagnosis. For example, the diagnosis unit applies different diagnosis methods according to the employee's position or department during diagnosis. The diagnosis unit can use AI to apply diagnosis methods tailored to the position or department. For example, the diagnosis unit distinguishes between statements made by superiors and subordinates according to the employee's position and applies appropriate diagnosis methods. In addition, the diagnosis unit can distinguish between statements made by the technical department and the sales department according to the employee's department and apply appropriate diagnosis methods. Furthermore, the diagnosis unit can prioritize the diagnosis of statements related to specific business content according to the employee's position or department. By applying diagnosis methods according to the employee's position or department, more appropriate diagnosis becomes possible. Specifically, the diagnosis unit refers to an employee information database (e.g., structured data including employee ID, position, department, business content, etc.) and performs diagnosis processing by linking it with statement data. The diagnosis unit extracts position information (e.g., manager, general staff, leader) and department information (e.g., technical department, sales department, administration department) as features and reflects them in the selection of diagnosis algorithms and parameter settings. For example, for statements from superiors to subordinates, a personality diagnosis model that considers power balance (e.g., SVM with inter-position weighting) is applied. For statements from the technical department, a context analysis model utilizing a technical term dictionary (e.g., custom BERT) is used. For statements from the sales department, a phrase detection algorithm specific to customer interactions (e.g., N-gram based) is applied. The diagnosis unit prioritizes the extraction and detailed diagnosis of statements related to business content (e.g., project progress, deadline adjustment). Examples of output from the diagnosis unit include “Statement ID: 78901, Position: Manager, Department: Sales, Diagnosis Model: Sales-specialized, Diagnosis Result: Cooperative type” and “Statement ID: 78902, Position: General staff, Department: Technical, Diagnosis Model: Technical term enhanced, Diagnosis Result: Logical type”. The diagnosis unit links these outputs to human resource management systems and harassment countermeasure dashboards to improve overall system accuracy. Unlike conventional uniform diagnosis processing or manual consideration of position and department, the diagnosis unit automatically utilizes position and department information in a high-dimensional feature space and dynamically switches diagnosis methods, thereby achieving technical effects such as improved diagnosis accuracy, reduced misdiagnosis rate, and flexible response to business characteristics. Application fields include corporate harassment countermeasures, organizational risk management, human resource management, and business process optimization.

The diagnosis unit can estimate the user's emotion and adjust the display method of the diagnosis result based on the estimated emotion of the user. For example, the diagnosis unit estimates the user's emotion and adjusts the display method of the diagnosis result based on the estimated emotion. The diagnosis unit is implemented using emotion estimation functions such as emotion engines or generative AI. Generative AI may include text generation AI (e.g., LLM) or multimodal generative AI, but is not limited to these examples. For instance, when the user is feeling stressed, the diagnosis unit provides a simple and highly visible display method. When the user is relaxed, the diagnosis unit can provide a display method that includes detailed information. Furthermore, when the user is excited, the diagnosis unit can provide a visually stimulating display method. By adjusting the display method of the diagnosis result based on the user's emotion, a display that is easy for the user to view becomes possible. Specifically, the diagnosis unit receives the user's statement text or audio data as input and uses an emotion estimation model (e.g., Transformer-based large language model or speech emotion recognition model) to output emotion categories (e.g., stress, relaxation, excitement) and emotion intensity scores (e.g., 0.85/1.0). The diagnosis unit dynamically adjusts display parameters such as dashboard or notification screen UI layout, color scheme, amount of information, and graph type according to the estimated emotion. For example, in a stress state, only important information is emphasized and the color tone is set to a calm scheme. In a relaxed state, detailed diagnosis results and history graphs are displayed. In an excited state, interactive displays using animation and accent colors are provided. Examples of output from the diagnosis unit include “User ID: 123, Emotion: Stress, Display Mode: Simple” and “User ID: 124, Emotion: Relaxation, Display Mode: Detailed”. The diagnosis unit links these outputs to the frontend display module to optimize the user experience. Unlike conventional uniform information display or manual adjustment by humans, the diagnosis unit automatically optimizes emotion estimation and display parameters, thereby achieving technical effects such as improved usability, reduced stress from information overload, and realization of context-adaptive UI. Application fields include corporate harassment countermeasure dashboards, employee mental health support systems, and customer support analysis tools.

The diagnosis unit can perform diagnosis based on the employee's geographic location information during diagnosis. For example, the diagnosis unit performs diagnosis based on the employee's geographic location information during diagnosis. The diagnosis unit can use AI to perform diagnosis that takes geographic location information into account. For instance, the diagnosis unit prioritizes the diagnosis of statements related to specific regions based on the employee's geographic location information. The diagnosis unit can also perform diagnosis that reflects the characteristics of each region by considering the employee's geographic location information. Furthermore, the diagnosis unit can diagnose statement patterns in specific regions based on the employee's geographic location information. By considering the employee's geographic location information, diagnosis that reflects regional characteristics becomes possible. Specifically, the diagnosis unit acquires employee location information data (e.g., structured data including GPS coordinates, office ID, city name, etc.) linked with statement data. The diagnosis unit extracts geographic features (e.g., city, country, office classification) and reflects them in the parameters and model selection of the diagnosis algorithm. For example, statement patterns frequently occurring in specific regions (e.g., dialects or local expressions in the Kansai office) are detected, and region-specialized natural language processing models (e.g., BERT incorporating regional dictionaries) are applied. The diagnosis unit automatically aggregates regional risk trends (e.g., increased stress statements in urban areas, increased cooperative statements in regional offices) and reflects them in the diagnosis results. Examples of output from the diagnosis unit include “Statement ID: 34567, Location: Tokyo Headquarters, Diagnosis Model: Urban type, Diagnosis Result: Logical type” and “Statement ID: 34568, Location: Osaka Branch, Diagnosis Model: Kansai-specialized, Diagnosis Result: Cooperative type”. The diagnosis unit links these outputs to human resource management systems and harassment countermeasure dashboards and utilizes them for planning organizational improvement measures that take regional characteristics into account. Unlike conventional uniform diagnosis processing or manual consideration of regional characteristics, the diagnosis unit automatically utilizes geographic location information in a high-dimensional feature space and dynamically switches region-specialized models, thereby achieving technical effects such as improved diagnosis accuracy, early detection of regional risks, and cross-organizational data utilization. Application fields include corporate multi-site harassment countermeasures, global human resource management, and region-specific risk management.

The diagnosis unit can analyze the employee's SNS activity during diagnosis and diagnose related statements. For example, the diagnosis unit analyzes the employee's SNS activity during diagnosis and diagnoses related statements. The diagnosis unit can use AI to perform diagnosis based on SNS activity. For instance, the diagnosis unit diagnoses statements related to specific topics based on the employee's SNS activity. The diagnosis unit can also analyze the employee's SNS activity and diagnose changes in the tone or emotion of statements. Furthermore, the diagnosis unit can diagnose specific keywords or hashtags based on the employee's SNS activity. By analyzing the employee's SNS activity, the diagnosis unit can diagnose related statements and perform more detailed diagnosis. Specifically, the diagnosis unit collects employee SNS post data (e.g., structured data including post ID, post text, timestamp, hashtag, emotion score, etc.) and integrates it with statement data for diagnosis processing. The diagnosis unit automatically executes topic classification on SNS (e.g., topic estimation by LDA), keyword extraction (e.g., TF-IDF score), hashtag frequency analysis, and tone change (e.g., positive/negative judgment). The diagnosis unit performs correlation analysis between SNS activity and internal statements (e.g., when negative posts increase on SNS, internal statements also tend to be aggressive) and dynamically adjusts parameters of the diagnosis algorithm (e.g., weighting of diagnosis items, weighting of emotion estimation models). Examples of output from the diagnosis unit include “Employee D: SNS Topic: Delayed delivery, Internal Statement: Aggressive, Diagnosis Result: Logical type” and “Employee E: SNS Hashtag: #WorkStyleReform, Internal Statement: Cooperative, Diagnosis Result: Cooperative type”. The diagnosis unit links these outputs to human resource management systems and harassment countermeasure dashboards and utilizes them for organizational risk analysis and human resource management that reflect SNS activity. Unlike conventional diagnosis targeting only internal data or manual SNS monitoring by humans, the diagnosis unit automatically integrates SNS activity in a high-dimensional feature space and dynamically optimizes the diagnosis algorithm, thereby achieving technical effects such as improved diagnosis accuracy, early risk detection, and cross-organizational data utilization. Application fields include corporate harassment countermeasures, SNS risk management, human resource management, and brand image protection.

The system according to the embodiment is not limited to the above examples and can be variously modified as follows, for example. Specifically, the system allows for diverse variations in the module configuration of the analysis unit, detection unit, and diagnosis unit, data flow, AI model architecture, types of input data, output formats, and algorithm parameter settings. For example, in the analysis unit, not only Transformer-based large language models but also different natural language processing models such as bidirectional LSTM, convolutional neural networks, or graph neural networks can be selectively installed. Input data may include text data (UTF-8 encoded string arrays, up to 4096 tokens per sample), audio data (WAV format sampled at 16 kHz, up to 60 seconds per utterance), image data (PNG format screenshots, 1280×720 pixels per image), as well as video data and IoT sensor logs. The preprocessing unit can combine various preprocessing methods such as noise removal, tokenization, speech-to-text conversion (ASR), removal of unnecessary words, normalization, and data augmentation (e.g., paraphrase generation, audio pitch conversion). In the detection unit, thresholds for risk statement detection and rule-based filter settings can be dynamically changed, and multiple AI models (e.g., multilayer perceptron+decision tree ensemble) can be used to improve judgment accuracy. In the diagnosis unit, personality diagnosis models such as multilayer perceptron, random forest, SVM, or clustering algorithms (e.g., K-means) can be combined to evaluate employees' intellectual personality and communication style from multiple perspectives. Furthermore, output data from each unit can be linked via API to various external systems such as human resource management systems, harassment countermeasure dashboards, and organizational improvement planning tools. These modifications enable the system to be flexibly customized according to company size, industry, organizational culture, and operational policies, and provide technical effects such as improved analysis accuracy, reduced false detection rate, optimized operational load, and efficient data management. Application fields include corporate harassment countermeasures, human resource management, internal communication analysis, risk management, mental health support, customer support analysis, and brand image protection.

The analysis unit can take into account the time zone of statements when analyzing the content of employee statements. For example, the analysis unit can distinguish between statements made during working hours and those made outside working hours and analyze them separately. The analysis unit can also analyze trends in statements during specific time zones and grasp statement patterns for each time zone. Furthermore, the analysis unit can analyze how employee statements change before and after specific events or meetings. By considering the time zone of statements, more detailed analysis becomes possible. Specifically, the analysis unit attaches timestamp information (e.g., ISO8601 format date and time, statement ID, event ID) to statement data and collaborates with a time-series database to manage the occurrence time of statements with high accuracy. The analysis unit extracts time zone features (e.g., working hours: 9:00-18:00, late night: 22:00-6:00, before/after events: 30 minutes before/after meeting start) and inputs them into natural language processing models (e.g., Transformer-based large language models or time-series LSTM). The analysis unit automatically aggregates statement trends for each time zone (e.g., increase in aggressive statements during late night, increase in stress statements immediately after meetings) and dynamically adjusts algorithm parameters for risk statement detection and personality diagnosis (e.g., threshold for risk score, weighting of emotion estimation models). Examples of input include “Text data stating ‘I can't take it anymore’ made at 23:15 on 2024-06-01” or “Statement ‘The deadline is tough’ made immediately after a meeting”. From these inputs, the analysis unit outputs time zone-dependent risk scores (e.g., +0.1 adjustment for late night), event-related emotion fluctuation patterns (e.g., stress score spikes before/after meetings), etc. These outputs are linked to the detection unit and diagnosis unit, enabling flexible analysis, detection, and diagnosis according to time zone and event. Unlike conventional uniform statement analysis or manual consideration of time zones by humans, the analysis unit automatically utilizes time-series information in a high-dimensional feature space and dynamically optimizes algorithms, thereby achieving technical effects such as improved analysis accuracy, early detection of abnormal statements, and visualization of organizational event risks. Application fields include corporate harassment countermeasures, mental health support for night shift workers, event risk management, and internal communication analysis.

The detection unit can take into account the frequency of statements when detecting the content of employee statements. For example, if specific aggressive statements are frequently made, the detection unit can evaluate the risk of those statements as high. The detection unit can also focus on monitoring statements from employees who have made multiple aggressive statements in a short period. Furthermore, if specific phrases or words are frequently used, the detection unit can analyze the usage of those phrases or words in detail. By considering the frequency of statements, more appropriate detection becomes possible. Specifically, the detection unit refers to a statement history database (e.g., structured data including statement ID, statement text, timestamp, speaker ID, emotion score) and automatically aggregates statement frequency over a certain period (e.g., past 7 days, 30 days). The detection unit extracts the occurrence count of aggressive words or phrases (e.g., ‘useless’ appearing 5 times in one week), frequency of aggressive statements per speaker (e.g., Employee A made 4 aggressive statements in 3 days) as features and inputs them into AI models (e.g., multilayer perceptron, decision tree, SVM). The detection unit dynamically adjusts the weighting of risk scores and detection thresholds according to statement frequency (e.g., lower threshold and increase detection sensitivity when frequency is high). Examples of input include “Text data where Employee B said ‘incompetent’ three times in one day” or “Statement records where the same phrase was repeated multiple times during a specific meeting”. From these inputs, the detection unit outputs frequency-dependent risk statement labels (e.g., risk level: high for frequency of 5 or more), enhanced monitoring flags (e.g., automatic notification when frequent in a short period), etc. These outputs are linked to database records and administrator notification modules and are used for early detection and focused monitoring of risk statements within the organization. Unlike conventional single-statement detection or manual frequency monitoring by humans, the detection unit automatically aggregates frequency information in a high-dimensional feature space and optimizes algorithms, thereby achieving technical effects such as improved detection accuracy, early detection of risk statements, and efficient organizational monitoring. Application fields include corporate harassment countermeasures, employee behavior monitoring, risk management, and early detection of organizational troubles.

The diagnosis unit can take into account the length of statements when diagnosing the content of employee statements. For example, the diagnosis unit can distinguish between long and short statements and analyze the characteristics of each. The diagnosis unit can also analyze the communication style of employees who frequently make long statements in detail. Furthermore, the diagnosis unit can focus on analyzing the content of short statements made by employees who frequently make short statements and diagnose their personality or intellectual characteristics. By considering the length of statements, more appropriate diagnosis becomes possible. Specifically, the diagnosis unit automatically extracts length indicators such as character count, word count, and sentence count (e.g., average word count per statement, maximum sentence length, short statement rate) from statement text data and manages them chronologically in collaboration with a statement history database (e.g., structured data including statement ID, statement text, timestamp, statement length, emotion score). The diagnosis unit extracts tendencies for long statements (e.g., 100 words or more per statement, 5 or more times per week) and short statements (e.g., less than 10 words per statement, 80% of all statements) as features and inputs them into personality diagnosis models (e.g., multilayer perceptron, random forest, SVM). The diagnosis unit dynamically adjusts diagnosis algorithm parameters according to statement length (e.g., increase weighting for logical/analytical types for long statement makers, increase weighting for intuitive/action types for short statement makers). Examples of input include “Text data where Employee C posted a long statement of 300 characters in one instance” or “Record where Employee D made 10 short statements of less than 10 words each in one day”. From these inputs, the diagnosis unit outputs statement length-dependent personality categories (e.g., long statement tendency: logical type, short statement tendency: action type), communication style diagnosis (e.g., frequent long statements=explanatory type, frequent short statements=directive type), etc. These outputs are linked to human resource management systems and organizational improvement planning tools and are used for appropriate employee placement and communication improvement. Unlike conventional uniform statement diagnosis or manual evaluation of statement length by humans, the diagnosis unit automatically utilizes statement length information in a high-dimensional feature space and dynamically optimizes diagnosis algorithms, thereby achieving technical effects such as improved diagnosis accuracy, optimal placement of human resources, and understanding of communication diversity within the organization. Application fields include corporate human resource management, internal communication analysis, mental health support, and risk management.

The analysis unit can take into account the tone of statements when analyzing the content of employee statements. For example, the analysis unit can determine whether the tone of a statement is aggressive or friendly and adjust the analysis result accordingly. The analysis unit can also focus on analyzing statements from employees who frequently speak in an aggressive tone. Furthermore, the analysis unit can analyze how the tone of statements changes over time and evaluate the content of statements based on changes in tone. By considering the tone of statements, more detailed analysis becomes possible. Specifically, the analysis unit applies emotion estimation models (e.g., Transformer-based large language models, speech emotion recognition models, BERT with emotion classification head) to statement text data or audio data and automatically extracts tone categories (e.g., aggressive, friendly, neutral, cooperative) and emotion intensity scores (e.g., aggressiveness 0.85/1.0, friendliness 0.15/1.0). The analysis unit collaborates with a statement history database (e.g., structured data including statement ID, statement text, timestamp, tone score) and analyzes time-series changes in tone (e.g., increasing aggressiveness in the last 10 statements). The analysis unit dynamically adjusts algorithm parameters for risk statement detection and personality diagnosis based on tone information (e.g., lower risk score threshold when aggressive tone is frequent, apply standard mode when friendly tone is frequent). Examples of input include “Text data where Employee E said ‘Don't mess with me’ (aggressive tone)” or “Audio data where Employee F said ‘Thank you always’ (friendly tone)”. From these inputs, the analysis unit outputs tone-dependent risk statement labels and personality diagnosis results. These outputs are linked to the detection unit and diagnosis unit and are used for early detection of risk statements and communication trends within the organization. Unlike conventional simple word matching or subjective tone evaluation by humans, the analysis unit automatically extracts tone information in a high-dimensional feature space and optimizes algorithms, thereby achieving technical effects such as improved analysis accuracy, reduced false detection rate, and healthier internal communication. Application fields include corporate harassment countermeasures, organizational risk management, human resource management, and mental health support.

The detection unit can take into account background information of statements when detecting the content of employee statements. For example, the detection unit can evaluate the risk of a statement by considering the situation or context in which the statement was made. The detection unit can also focus on analyzing statements made before or after specific events or meetings, taking background information into account. Furthermore, the detection unit can evaluate the risk of statements by considering the place or time zone in which the statement was made. By considering background information of statements, more appropriate detection becomes possible. Specifically, the detection unit attaches background information such as event ID, meeting ID, location information (e.g., office ID, meeting room name, remote/on-site classification), and timestamp to statement data and manages multidimensional context information in collaboration with a statement history database. The detection unit extracts background information as features and inputs them into AI models (e.g., multilayer perceptron, decision tree, SVM). For example, for statements made immediately after meetings or before/after specific events, the weighting of risk scores is strengthened to enable early detection of risk statements. Based on location information, the detection unit analyzes statements made in remote work environments or at specific sites and dynamically adjusts detection algorithm parameters (e.g., thresholds, feature weighting). Examples of input include “Text data stating ‘The deadline is tough’ made immediately after a meeting at 10:00 on 2024-06-01” or “Audio data stating ‘I'm at my limit’ made during a remote meeting”. From these inputs, the detection unit outputs background information-dependent risk statement labels and notification priorities. These outputs are linked to database records and administrator notification modules and are used for early detection and focused monitoring of risk statements within the organization. Unlike conventional detection based only on statement content or manual situation judgment by humans, the detection unit automatically utilizes background information in a high-dimensional feature space and optimizes algorithms, thereby achieving technical effects such as improved detection accuracy, early detection of risk statements, and efficient organizational monitoring. Application fields include corporate harassment countermeasures, event risk management, remote work environment monitoring, and early detection of organizational troubles.

The analysis unit can estimate the user's emotion and adjust the priority of analysis based on the estimated emotion of the user. For example, when the user is feeling stressed, the analysis unit can prioritize the analysis of statements with high importance. When the user is relaxed, the analysis unit can analyze the overall content of statements in a balanced manner. Furthermore, when the user is excited, the analysis unit can focus on analyzing specific statements by considering emotional fluctuations. By adjusting the priority of analysis based on the user's emotion, more appropriate analysis becomes possible. Specifically, the analysis unit receives the user's statement text data or audio data as input and uses an emotion estimation model (e.g., Transformer-based large language model, speech emotion recognition model) to output emotion categories (e.g., stress, relaxation, excitement) and emotion intensity scores (e.g., stress 0.85/1.0). The analysis unit dynamically adjusts the priority parameters for statement analysis according to the estimated emotion score (e.g., prioritize aggressive or negative statements in a stress state, balanced analysis in a relaxed state, focus on emotional fluctuation statements in an excited state). Examples of input include “Text data where the user said ‘I'm at my limit’ (high stress)” or “Audio data with heightened voice during a meeting (excited state)”. From these inputs, the analysis unit outputs a prioritized list of statements for analysis (e.g., prioritize stress statements), analysis mode (e.g., detailed, standard, focused), etc. These outputs are linked to the detection unit and diagnosis unit and are used for early detection of risk statements and important statements within the organization. Unlike conventional uniform statement analysis or subjective prioritization by humans, the analysis unit automatically utilizes emotional information in a high-dimensional feature space and dynamically optimizes analysis priority, thereby achieving technical effects such as improved analysis accuracy, early detection of risk statements, and efficient use of computational resources. Application fields include corporate harassment countermeasures, employee mental health support, internal communication analysis, and risk management.

The detection unit can estimate the user's emotion and adjust the timing of detection based on the estimated emotion of the user. For example, when the user is feeling stressed, the detection unit can increase the frequency of detection and perform more detailed detection. When the user is relaxed, the detection unit can adjust the frequency of detection and perform only the minimum necessary detection. Furthermore, when the user is excited, the detection unit can adjust the timing of detection and perform detection that takes emotional fluctuations into account. By adjusting the timing of detection based on the user's emotion, more appropriate detection becomes possible. Specifically, the detection unit receives the user's statement text data or audio data as input and uses an emotion estimation model (e.g., Transformer-based large language model, speech emotion recognition model) to output emotion categories (e.g., stress, relaxation, excitement) and emotion intensity scores (e.g., stress 0.85/1.0). The detection unit dynamically adjusts detection frequency parameters according to the estimated emotion score (e.g., detect every hour in a stress state, detect once a day in a relaxed state, detect immediately upon emotional fluctuation in an excited state). Examples of input include “Text data where the user said ‘I'm at my limit’ (high stress)” or “Audio data with heightened voice during a meeting (excited state)”. From these inputs, the detection unit outputs detection timing (e.g., immediate, periodic, upon fluctuation), detection mode (e.g., detailed, standard), etc. These outputs are linked to database records and administrator notification modules and are used for early detection of risk statements and important statements within the organization. Unlike conventional uniform detection timing or subjective judgment by humans, the detection unit automatically utilizes emotional information in a high-dimensional feature space and dynamically optimizes detection timing, thereby achieving technical effects such as improved detection accuracy, early detection of risk statements, and efficient use of computational resources. Application fields include corporate harassment countermeasures, employee mental health support, internal risk management, and customer support analysis.

The diagnosis unit can estimate the user's emotion and adjust the priority of diagnosis based on the estimated emotion of the user. For example, when the user is feeling stressed, the diagnosis unit can prioritize the analysis of high-importance diagnosis items. When the user is relaxed, the diagnosis unit can analyze all diagnosis items in a balanced manner. Furthermore, when the user is excited, the diagnosis unit can focus on analyzing specific diagnosis items by considering emotional fluctuations. By adjusting the priority of diagnosis based on the user's emotion, more appropriate diagnosis becomes possible. Specifically, the diagnosis unit receives the user's statement text data or audio data as input and uses an emotion estimation model (e.g., Transformer-based large language model, speech emotion recognition model) to output emotion categories (e.g., stress, relaxation, excitement) and emotion intensity scores (e.g., stress 0.85/1.0). The diagnosis unit dynamically adjusts priority parameters for diagnosis items according to the estimated emotion score (e.g., prioritize mental health diagnosis and aggressiveness diagnosis in a stress state, balanced diagnosis in a relaxed state, focus on emotional fluctuation diagnosis in an excited state). Examples of input include “Text data where the user said ‘I'm at my limit’ (high stress)” or “Audio data with heightened voice during a meeting (excited state)”. From these inputs, the diagnosis unit outputs a prioritized list of diagnosis items (e.g., prioritize stress diagnosis), diagnosis mode (e.g., detailed, standard, focused), etc. These outputs are linked to human resource management systems and harassment countermeasure dashboards and are used for early detection of risk diagnosis and important diagnosis within the organization. Unlike conventional uniform diagnosis item analysis or subjective prioritization by humans, the diagnosis unit automatically utilizes emotional information in a high-dimensional feature space and dynamically optimizes diagnosis priority, thereby achieving technical effects such as improved diagnosis accuracy, early detection of risk diagnosis, and efficient use of computational resources. Application fields include corporate human resource management, employee mental health support, internal communication analysis, and risk management.

The analysis unit can estimate the user's emotion and adjust the notification method of the analysis result based on the estimated emotion of the user. For example, when the user is feeling stressed, the analysis unit can provide a simple and highly visible notification method. When the user is relaxed, the analysis unit can provide a notification method that includes detailed information. Furthermore, when the user is excited, the analysis unit can provide a visually stimulating notification method. By adjusting the notification method of the analysis result based on the user's emotion, notifications that are easy for the user to view become possible. Specifically, the analysis unit receives the user's statement text or audio data as input and uses an emotion estimation model (e.g., Transformer-based large language model or speech emotion recognition model) to output emotion categories (e.g., stress, relaxation, excitement) and emotion intensity scores (e.g., 0.85/1.0). The analysis unit dynamically adjusts notification parameters such as notification UI layout, color scheme, amount of information, notification frequency, and graph type according to the estimated emotion. For example, in a stress state, only important information is emphasized and a simple notification with a calm color scheme is provided. In a relaxed state, notifications including detailed analysis results and history graphs are provided. In an excited state, interactive notifications using animation and accent colors are provided. Examples of output from the analysis unit include “User ID: 123, Emotion: Stress, Notification Mode: Simple” and “User ID: 124, Emotion: Relaxation, Notification Mode: Detailed”. The analysis unit links these outputs to the frontend notification module to optimize the user experience. Unlike conventional uniform notification methods or manual adjustment by humans, the analysis unit automatically optimizes emotion estimation and notification parameters, thereby achieving technical effects such as improved usability, reduced stress from information overload, and realization of context-adaptive notification UI. Application fields include corporate harassment countermeasure dashboards, employee mental health support systems, and customer support analysis tools.

The detection unit can estimate the user's emotion and adjust the notification method of the detection result based on the estimated emotion of the user. For example, when the user is feeling stressed, the detection unit can provide a simple and highly visible notification method. When the user is relaxed, the detection unit can provide a notification method that includes detailed information. Furthermore, when the user is excited, the detection unit can provide a visually stimulating notification method. By adjusting the notification method of the detection result based on the user's emotion, notifications that are easy for the user to view become possible. Specifically, the detection unit receives the user's statement text or audio data as input and uses an emotion estimation model (e.g., Transformer-based large language model or speech emotion recognition model) to output emotion categories (e.g., stress, relaxation, excitement) and emotion intensity scores (e.g., 0.85/1.0). The detection unit dynamically adjusts notification parameters such as notification UI layout, color scheme, amount of information, notification frequency, and graph type according to the estimated emotion. For example, in a stress state, only important information is emphasized and a simple notification with a calm color scheme is provided. In a relaxed state, notifications including detailed detection results and history graphs are provided. In an excited state, interactive notifications using animation and accent colors are provided. Examples of output from the detection unit include “User ID: 123, Emotion: Stress, Notification Mode: Simple” and “User ID: 124, Emotion: Relaxation, Notification Mode: Detailed”. The detection unit links these outputs to the frontend notification module to optimize the user experience. Unlike conventional uniform notification methods or manual adjustment by humans, the detection unit automatically optimizes emotion estimation and notification parameters, thereby achieving technical effects such as improved usability, reduced stress from information overload, and realization of context-adaptive notification UI. Application fields include corporate harassment countermeasure dashboards, employee mental health support systems, and customer support analysis tools.

Below is a brief explanation of the processing flow of Example of the Embodiment. Specifically, the system implements a series of data flows from acquisition of internal communication data, analysis, detection of risk statements, intellectual personality diagnosis, to output linkage. In Step 1, the data acquisition module collects various internal communication data such as internal chat, email, voice conversation logs, and image data (e.g., UTF-8 encoded string arrays, 16 kHz sampled WAV audio, PNG format screenshots), and the preprocessing unit performs preprocessing such as noise removal, tokenization, speech-to-text conversion (ASR), removal of unnecessary words, and normalization. In Step 2, the analysis unit uses natural language processing models such as Transformer-based large language models, bidirectional LSTM, and convolutional neural networks to extract word embedding vectors and contextual features, and outputs lists of aggressive words and context-dependent risk scores. Examples of input to the analysis unit include “Text data where a superior said to a subordinate ‘You are useless’” or “Statement records extracted from audio data during a meeting”. In Step 3, the detection unit receives the output from the analysis unit, performs threshold judgment and rule-based filtering, and records the detection results (e.g., risk statement label, speaker ID, timestamp, risk score) as structured data in the database. Examples of output from the detection unit include “Statement ID: 12345, Risk Statement: True, Score: 0.92, Speaker: Employee A, Date: 2024-06-01 10:15”. In Step 4, the diagnosis unit inputs statement history and tone information accumulated by the detection unit as time-series vectors and uses a personality diagnosis model (e.g., multilayer perceptron for Big Five personality trait estimation) to classify employees into multiple intellectual personality categories. Examples of output from the diagnosis unit include “Employee A: High extraversion, low agreeableness, intellectual characteristic: logical type”. These outputs are automatically linked to human resource management systems and harassment countermeasure dashboards and are used for early intervention by administrators and planning of organizational improvement measures. Unlike conventional visual audits or questionnaire aggregation by humans, the system uses computer-specific unconventional methods such as automatic analysis in high-dimensional feature space, hybrid judgment of rule-based and machine learning, and automatic detection of time-series changes, thereby achieving technical effects such as significant improvement in processing speed, reduction of false detection rate, and efficient data management. Application fields include corporate harassment countermeasures, appropriate placement of human resources, healthy internal communication, risk management, and employee mental health support.

Step 1: The analysis unit reads internal communications and analyzes words or surrounding context. For example, the analysis unit analyzes the content of employee statements and checks whether specific words or phrases are included. The analysis unit can analyze the context of statements using natural language processing technology. Step 2: The detection unit detects statements with potential risk based on the content analyzed by the analysis unit and accumulates them in a database. For example, the detection unit detects aggressive statements and accumulates them in the database. The detection unit can evaluate the risk of statement content using AI. Step 3: The diagnosis unit performs intellectual personality diagnosis based on the data accumulated by the detection unit and categorizes employees. For example, the diagnosis unit analyzes the content or tone of employee statements and diagnoses the personality or intellectual characteristics of the employee. The diagnosis unit can evaluate the intellectual personality of statement content using AI. Specifically, in Step 1, the analysis unit collects various internal communication data such as internal chat, email, voice conversation logs, and image data (e.g., UTF-8 encoded string arrays, 16 kHz sampled WAV audio, PNG format screenshots) using the data acquisition module, and the preprocessing unit performs preprocessing such as noise removal, tokenization, speech-to-text conversion (ASR), removal of unnecessary words, and normalization. The analysis unit uses natural language processing models such as Transformer-based large language models, bidirectional LSTM, and convolutional neural networks to extract word embedding vectors and contextual features, and outputs lists of aggressive words and context-dependent risk scores. Examples of input to the analysis unit include “Text data where a superior said to a subordinate ‘You are useless’” or “Statement records extracted from audio data during a meeting”. From these inputs, the analysis unit outputs aggressive word lists (e.g., [‘useless’, ‘incompetent’, ‘fool’]) and context-dependent risk scores (e.g., 0.85/1.0). In Step 2, the detection unit receives the output from the analysis unit, performs threshold judgment and rule-based filtering, and records the detection results (e.g., risk statement label, speaker ID, timestamp, risk score) as structured data in the database. Examples of output from the detection unit include “Statement ID: 12345, Risk Statement: True, Score: 0.92, Speaker: Employee A, Date: 2024-06-01 10:15”. In Step 3, the diagnosis unit inputs statement history and tone information accumulated by the detection unit as time-series vectors and uses a personality diagnosis model (e.g., multilayer perceptron for Big Five personality trait estimation) to classify employees into multiple intellectual personality categories. Examples of output from the diagnosis unit include “Employee A: High extraversion, low agreeableness, intellectual characteristic: logical type”. These outputs are automatically linked to human resource management systems and harassment countermeasure dashboards and are used for early intervention by administrators and planning of organizational improvement measures. Unlike conventional visual audits or questionnaire aggregation by humans, the system uses computer-specific unconventional methods such as automatic analysis in high-dimensional feature space, hybrid judgment of rule-based and machine learning, and automatic detection of time-series changes, thereby achieving technical effects such as significant improvement in processing speed, reduction of false detection rate, and efficient data management. Application fields include corporate harassment countermeasures, appropriate placement of human resources, healthy internal communication, risk management, and employee mental health support.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the above-described analysis unit, detection unit, and diagnosis unit is implemented by at least one of, for example, a smart device 14 and a data processing apparatus 12. For example, the analysis unit is implemented by a control unit 46A of the smart device 14, analyzes the content of employee statements, and checks whether specific words or phrases are included. The detection unit is implemented, for example, by a specific processing unit 290 of the data processing apparatus 12, detects aggressive statements, and accumulates them in a database 24. The diagnosis unit is implemented, for example, by a specific processing unit 290 of the data processing apparatus 12, analyzes the content or tone of employee statements, and diagnoses the personality or intellectual characteristics of the employee. The correspondence between each unit and the apparatus or control unit is not limited to the examples described above and various modifications are possible.

### Second Embodiment

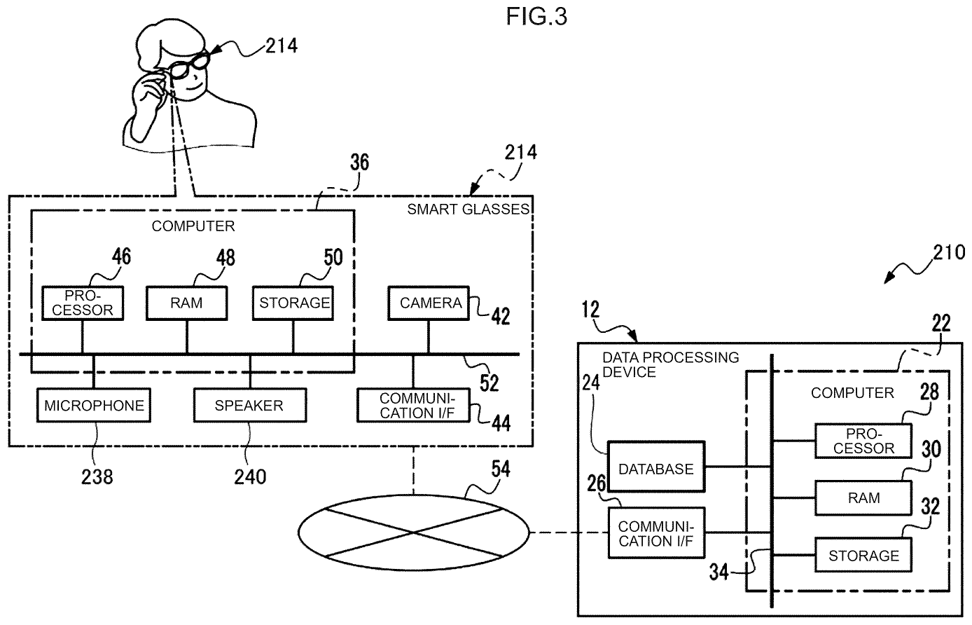

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

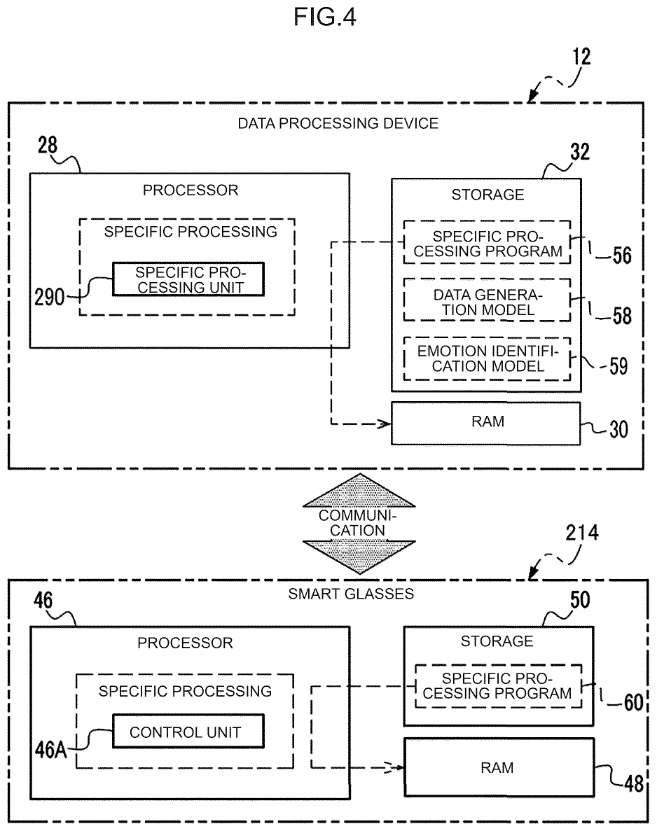

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the above-described analysis unit, detection unit, and diagnosis unit is implemented by at least one of, for example, smart glasses 214 and a data processing apparatus 12. For example, the analysis unit is implemented by a control unit 46A of the smart glasses 214, analyzes the content of employee statements, and checks whether specific words or phrases are included. The detection unit is implemented, for example, by a specific processing unit 290 of the data processing apparatus 12, detects aggressive statements, and accumulates them in a database 24. The diagnosis unit is implemented, for example, by a specific processing unit 290 of the data processing apparatus 12, analyzes the content or tone of employee statements, and diagnoses the personality or intellectual characteristics of the employee. The correspondence between each unit and the apparatus or control unit is not limited to the examples described above and various modifications are possible.

### Third Embodiment

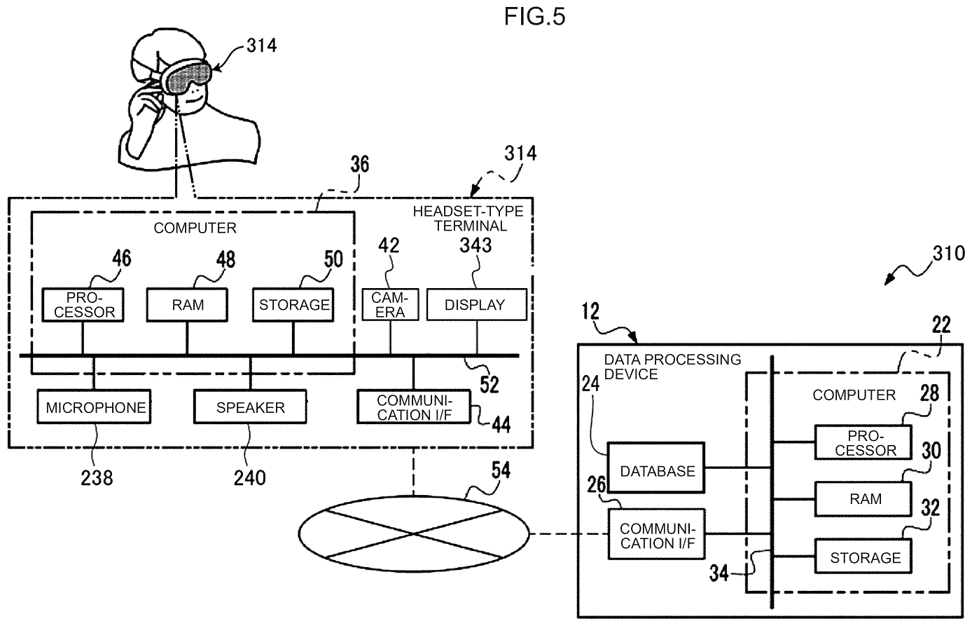

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

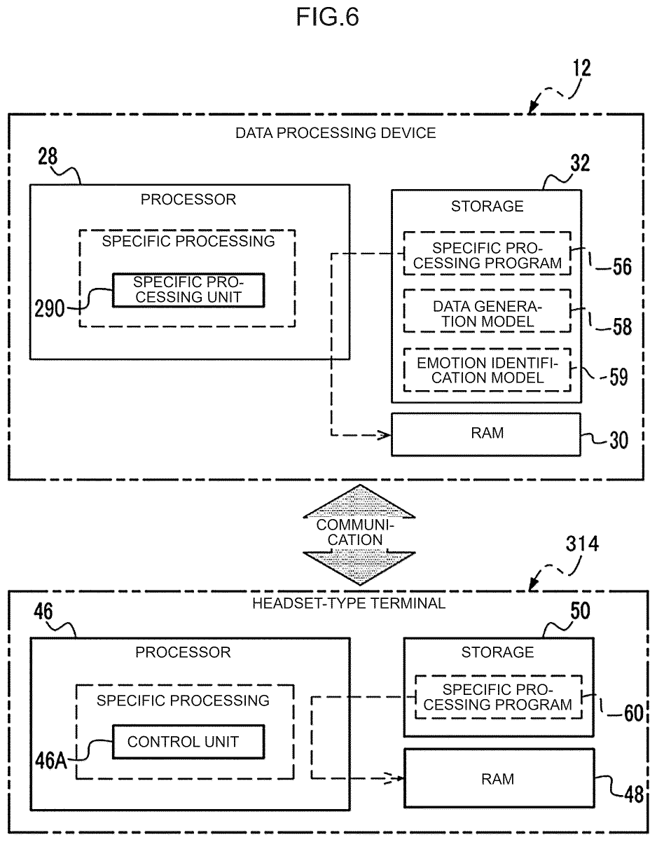

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the above-described analysis unit, detection unit, and diagnosis unit is implemented by at least one of, for example, a headset-type terminal 314 and a data processing apparatus 12. For example, the analysis unit is implemented by a control unit 46A of the headset-type terminal 314, analyzes the content of employee statements, and checks whether specific words or phrases are included. The detection unit is implemented, for example, by a specific processing unit 290 of the data processing apparatus 12, detects aggressive statements, and accumulates them in a database 24. The diagnosis unit is implemented, for example, by a specific processing unit 290 of the data processing apparatus 12, analyzes the content or tone of employee statements, and diagnoses the personality or intellectual characteristics of the employee. The correspondence between each unit and the apparatus or control unit is not limited to the examples described above and various modifications are possible.

### Fourth Embodiment

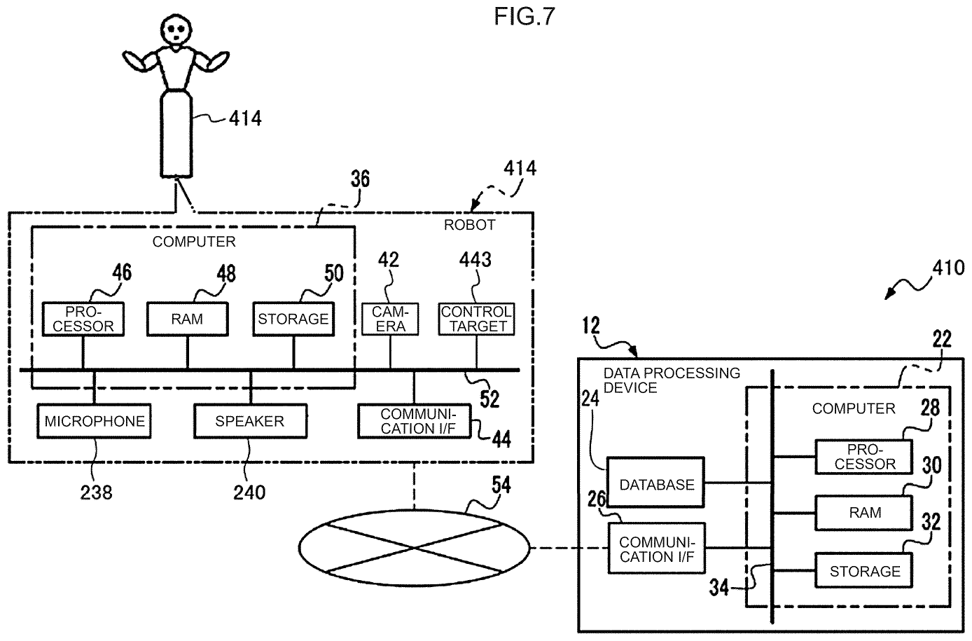

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

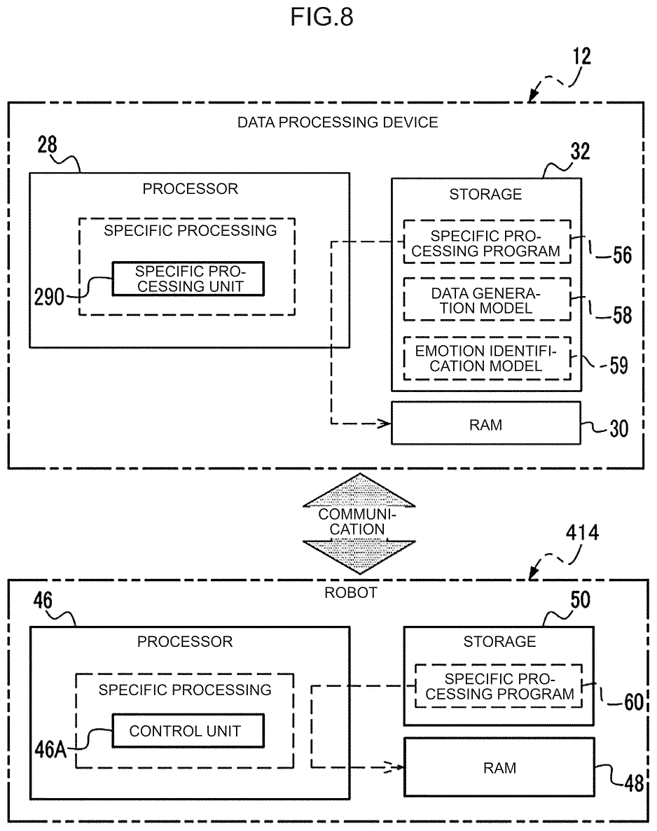

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the above-described analysis unit, detection unit, and diagnosis unit is implemented by at least one of, for example, a robot 414 and a data processing apparatus 12. For example, the analysis unit is implemented by a control unit 46A of the robot 414, analyzes the content of employee statements, and checks whether specific words or phrases are included. The detection unit is implemented, for example, by a specific processing unit 290 of the data processing apparatus 12, detects aggressive statements, and accumulates them in a database 24. The diagnosis unit is implemented, for example, by a specific processing unit 290 of the data processing apparatus 12, analyzes the content or tone of employee statements, and diagnoses the personality or intellectual characteristics of the employee. The correspondence between each unit and the apparatus or control unit is not limited to the examples described above and various modifications are possible.

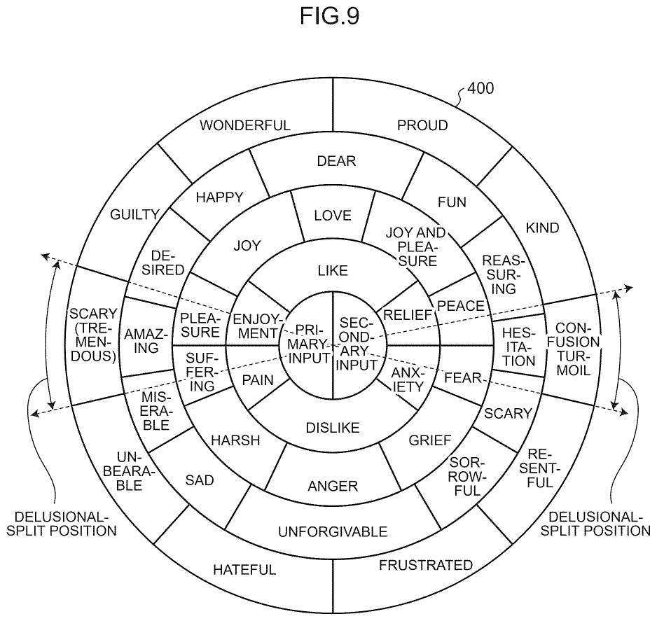

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

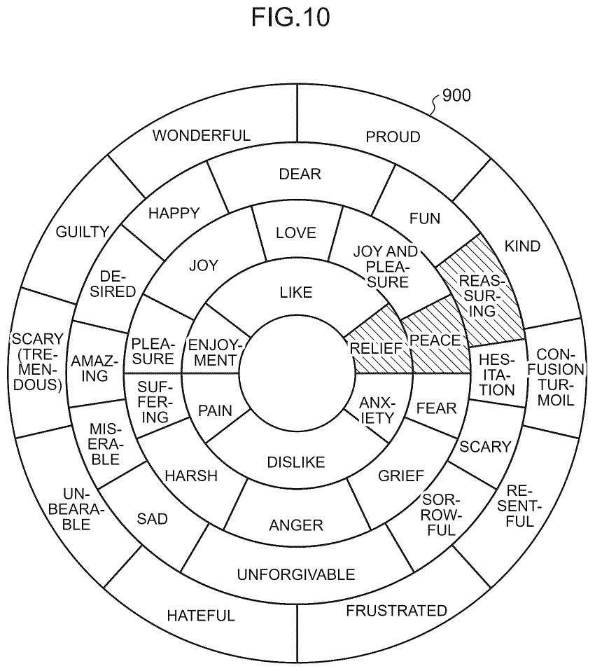

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: an analysis unit configured to read internal communications and analyze words or surrounding context; a detection unit configured to detect statements with potential risk based on the content analyzed by the analysis unit and accumulate them in a database; and a diagnosis unit configured to perform intellectual personality diagnosis and categorize employees based on the data accumulated by the detection unit.
(Supplementary Note 2) The system according to Supplementary Note 1, wherein the analysis unit analyzes the content of employee statements and checks whether specific words or phrases are included.
(Supplementary Note 3) The system according to Supplementary Note 1, wherein the detection unit detects specific aggressive statements and accumulates them in the database.
(Supplementary Note 4) The system according to Supplementary Note 1, wherein the diagnosis unit analyzes the content or tone of employee statements and diagnoses the personality or intellectual characteristics of the employee.
(Supplementary Note 5) The system according to Supplementary Note 1, wherein the analysis unit estimates the user's emotion and adjusts the accuracy of the analysis based on the estimated emotion of the user.
(Supplementary Note 6) The system according to Supplementary Note 1, wherein the analysis unit adjusts the analysis algorithm by referring to the employee's past statement history during analysis.
(Supplementary Note 7) The system according to Supplementary Note 1, wherein the analysis unit applies different analysis methods according to the employee's position or department during analysis.
(Supplementary Note 8) The system according to Supplementary Note 1, wherein the analysis unit estimates the user's emotion and adjusts the display method of the analysis result based on the estimated emotion of the user.
(Supplementary Note 9) The system according to Supplementary Note 1, wherein the analysis unit performs analysis based on the employee's geographic location information during analysis.
(Supplementary Note 10) The system according to Supplementary Note 1, wherein the analysis unit analyzes the employee's SNS activity during analysis and analyzes related statements.
(Supplementary Note 11) The system according to Supplementary Note 1, wherein the detection unit estimates the user's emotion and adjusts the detection criteria based on the estimated emotion of the user.
(Supplementary Note 12) The system according to Supplementary Note 1, wherein the detection unit adjusts the detection algorithm by referring to the employee's past statement history during detection.
(Supplementary Note 13) The system according to Supplementary Note 1, wherein the detection unit applies different detection methods according to the employee's position or department during detection.
(Supplementary Note 14) The system according to Supplementary Note 1, wherein the detection unit estimates the user's emotion and adjusts the display method of the detection result based on the estimated emotion of the user.
(Supplementary Note 15) The system according to Supplementary Note 1, wherein the detection unit performs detection based on the employee's geographic location information during detection.
(Supplementary Note 16) The system according to Supplementary Note 1, wherein the detection unit analyzes the employee's SNS activity during detection and detects related statements.
(Supplementary Note 17) The system according to Supplementary Note 1, wherein the diagnosis unit estimates the user's emotion and adjusts the accuracy of the diagnosis based on the estimated emotion of the user.
(Supplementary Note 18) The system according to Supplementary Note 1, wherein the diagnosis unit adjusts the diagnosis algorithm by referring to the employee's past statement history during diagnosis.
(Supplementary Note 19) The system according to Supplementary Note 1, wherein the diagnosis unit applies different diagnosis methods according to the employee's position or department during diagnosis.
(Supplementary Note 20) The system according to Supplementary Note 1, wherein the diagnosis unit estimates the user's emotion and adjusts the display method of the diagnosis result based on the estimated emotion of the user.
(Supplementary Note 21) The system according to Supplementary Note 1, wherein the diagnosis unit performs diagnosis based on the employee's geographic location information during diagnosis.
(Supplementary Note 22) The system according to Supplementary Note 1, wherein the diagnosis unit analyzes the employee's SNS activity during diagnosis and diagnoses related statements.

What is claimed is:

1. A system comprising:
circuitry configured to:
receive, from a client terminal via a communication interface and a packet-switched network, input data comprising at least one of text data or audio data;
extract, using a natural language processing model comprising a Transformer-based encoder, a feature vector of 768 or more dimensions from the input data;
compute a score by applying a classification model to the feature vector, and store, in a database, structured data comprising the score and a timestamp;
extract, from the structured data stored in the database, a time-series tensor comprising scores associated with a plurality of input data items; and
generate, by inputting the time-series tensor into a neural network comprising at least one of a multilayer perceptron or a recurrent neural network, a category label and a confidence score.

2. The system according to claim 1, wherein the input data comprises internal communication data of an organization, the internal communication data comprising at least one of chat messages, email messages, or voice conversation logs.

3. The system according to claim 1, wherein the circuitry is further configured to preprocess the input data by performing at least one of noise removal, tokenization, or speech-to-text conversion before extracting the feature vector.

4. The system according to claim 1, wherein the natural language processing model comprises a bidirectional encoder and generates the feature vector as a contextual embedding that captures semantic relationships between words in the input data.

5. The system according to claim 1, wherein the score comprises a risk score indicating a degree to which the input data satisfies a risk criterion, and wherein the circuitry is further configured to compare the risk score with a threshold to determine whether to store the structured data in the database.

6. The system according to claim 1, wherein the category label comprises a personality trait category based on a Big Five personality model, the personality trait category comprising at least one of extraversion, agreeableness, conscientiousness, neuroticism, or openness.

7. The system according to claim 1, wherein the circuitry is further configured to estimate an emotion of a user by applying an emotion identification model to the input data, and to adjust an accuracy parameter of the classification model based on the estimated emotion.

8. The system according to claim 7, wherein the circuitry is configured to increase a depth of feature extraction when the estimated emotion indicates stress, and to apply a simplified classification mode when the estimated emotion indicates relaxation.

9. The system according to claim 1, wherein the circuitry is further configured to retrieve, from the database, historical structured data associated with a user identifier, and to adjust a parameter of the classification model based on patterns extracted from the historical structured data.

10. The system according to claim 1, wherein the circuitry is further configured to receive attribute data associated with a source of the input data, the attribute data comprising at least one of a position level or a department identifier, and to select a classification model from a plurality of classification models based on the attribute data.

11. The system according to claim 1, wherein the circuitry is further configured to estimate an emotion of a user by applying an emotion identification model to the input data, and to adjust a threshold for storing the structured data in the database based on the estimated emotion.

12. The system according to claim 1, wherein the circuitry is further configured to receive geographic location information associated with the input data, and to select a natural language processing model from a plurality of natural language processing models based on the geographic location information.

13. The system according to claim 1, wherein the circuitry is further configured to receive social media activity data associated with a source of the input data, analyze the social media activity data using a topic classification model, and adjust a weighting parameter of the classification model based on topics extracted from the social media activity data.

14. The system according to claim 1, wherein the circuitry is further configured to extract a tone feature from the input data using an emotion estimation model, the tone feature comprising at least one of an aggressiveness score or a friendliness score, and to adjust the score based on the tone feature.

15. The system according to claim 1, wherein the circuitry is further configured to extract a timestamp from the input data, determine a time zone category based on the timestamp, and adjust a weighting parameter of the classification model based on the time zone category.

16. The system according to claim 1, wherein the circuitry is further configured to compute a frequency of input data items associated with a user identifier within a time window, and to adjust the threshold for storing the structured data based on the computed frequency.

17. The system according to claim 1, wherein the circuitry is further configured to estimate an emotion of a user by applying an emotion identification model to the input data, and to adjust a display parameter of an output transmitted to the client terminal based on the estimated emotion, the display parameter comprising at least one of a layout complexity, a color scheme, or an amount of information.

18. A system comprising:
a communication interface configured to communicate, via a packet-switched network conforming to at least one of a 5G, Wi-Fi, or Bluetooth communication standard, with a client terminal comprising a microphone, a speaker, a camera having a CMOS image sensor, and a display;
a processor;
a random-access memory;
a memory storing a natural language processing model comprising a Transformer-based encoder, a classification model, and an emotion identification model;
a database; and
circuitry configured to:
receive, from the client terminal via the communication interface, input data comprising at least one of text data or audio data captured by the microphone;
extract, using the natural language processing model, a feature vector of 768 or more dimensions from the input data;
compute a score by applying the classification model to the feature vector, and store, in the database, structured data comprising the score, a timestamp, and a source identifier;
estimate an emotion of a user by applying the emotion identification model to the input data, and adjust at least one of a threshold for storing the structured data or a parameter of the classification model based on the estimated emotion;
extract, from the structured data stored in the database, a time-series tensor comprising scores associated with a plurality of input data items; and
generate, by inputting the time-series tensor into a neural network comprising at least one of a multilayer perceptron or a recurrent neural network, a category label and a confidence score, and transmit the category label to the client terminal via the communication interface.

19. The system according to claim 18, wherein the memory further stores a data generation model comprising at least one of a text generation AI, an image generation AI, or a multimodal generation AI, and wherein the data generation model is a fine-tuned model configured to output inference results from prompts without instructions.

20. A method performed by circuitry of a system comprising a communication interface, a memory storing a natural language processing model comprising a Transformer-based encoder and a classification model, and a database, the method comprising:
receiving, from a client terminal via the communication interface and a packet-switched network, input data comprising at least one of text data or audio data;
extracting, using the natural language processing model, a feature vector of 768 or more dimensions from the input data;
computing a score by applying the classification model to the feature vector, and storing, in the database, structured data comprising the score and a timestamp;
extracting, from the structured data stored in the database, a time-series tensor comprising scores associated with a plurality of input data items; and
generating, by inputting the time-series tensor into a neural network comprising at least one of a multilayer perceptron or a recurrent neural network, a category label and a confidence score.
